SEQUENCE LISTING (8 sequences)

SEQ ID NO: 1 - protein, 107 aa, Homo sapiens
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIK

SEQ ID NO: 2 - protein, 119 aa, Homo sapiens
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSS

SEQ ID NO: 3 - protein, 107 aa, Homo sapiens [sequence omitted]

SEQ ID NO: 4 - protein, 113 aa, Homo sapiens [sequence omitted]

SEQ ID NO: 5 - protein, 9 aa, Homo sapiens
IMDQVPFSV

SEQ ID NO: 6 - protein, 9 aa, Homo sapiens
YLEPGPVTV

SEQ ID NO: 7 - protein, 335 aa, Homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 8 - protein, 288 aa, Homo sapiens
MQIPQAPWPVVWAVLQLGWRPGWFLDSPDRPWNPPTFFPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

(19) United States
(12) Patent Application Publication
(10) Pub. No.: US 2017/0355768 A1
(43) Pub. Date: Dec. 14, 2017

(54) COMBINATION OF ANTI-CS1 AND ANTI-PD1 ANTIBODIES TO TREAT CANCER (MYELOMA)

(71) Applicant: BRISTOL-MYERS SQUIBB COMPANY, Princeton, NJ (US)

(72) Inventors: Michael Darron ROBBINS, Whitehouse Station, NJ (US); Robert F. Graziano, Frenchtown, NJ (US); Natalie Bezman, Foster City, CA (US)

(21) Appl. No.: 15/531,538

(22) PCT Filed: Dec. 3, 2015
(86) PCT No.: PCT/US2015/063585

§ 371 Date: May 30, 2017

Related U.S. Application Data
(60) Provisional application No. 62/087,489, filed on Dec. 4, 2014.

(51) Int. Cl. C07K 16/28; A61K 39/00

(52) U.S. Cl. CPC C07K 16/2818; C07K 16/2803; C07K 2317/24; A61K 2039/507; C07K 2317/73; C07K 2317/21

(57) ABSTRACT

The invention described herein relates to therapeutic dosing regimens and combinations thereof for use in enhancing the therapeutic efficacy of anti-CS1 antibodies in combination with an anti-Programmed Death-1 (PD-1) antibody.

This application claims benefit to provisional application U.S. Ser. No. 62/087,489 filed Dec. 4, 2014 under 35 U.S.C. §119(e). The entire teachings of the referenced application are incorporated herein by reference.

### FIELD OF THE INVENTION

The invention described herein relates to therapeutic dosing regimens and combinations thereof for use in enhancing the therapeutic efficacy of anti-CS1 antibodies in combination with an anti-Programmed Death-1 (PD-1) antibody.

### BACKGROUND OF THE INVENTION

The National Cancer Institute has estimated that in the United States alone, 1 in 3 people will be struck with cancer during their lifetime. Moreover, approximately 50% to 60% of people contracting cancer will eventually succumb to the disease. The widespread occurrence of this disease underscores the need for improved anticancer regimens for the treatment of malignancy.

Cancer can occur in any tissue or organ of the body. Plasma cell neoplasms, including multiple myeloma, “Solitary” myeloma of bone, extramedullary plasmacytoma, plasma cell leukemia, macroglobulinemia (including Waldenstrom's macroglobulinemia), heavy-chain disease, primary amyloidosis, monoclonal gammopathy of unknown significance (MGUS) are associated with increased expression of immunoglobulins. Chronic lymphocytic leukemia (CLL), a non-plasma cell neoplasm, is also associated with high levels of immunoglobulin expression.

Increased expression of immunoglobulin can also be seen in malignant diseases. Like autoimmune disorders, the etiology of cancer is similarly multi-factorial in origin. Cancer, which is the second leading cause of death in the United States, has been linked to mutations in DNA that cause unrestrained growth of cells. Genetic predisposition plays a large role in the development of many cancers, combined with environmental factors, such as smoking and chemical mutagenesis.

Myelomas are tumors of plasma cells derived from a single clone, which typically originates in secondary lymphoid tissue and then migrates into and resides in bone marrow tissue. Myelomas commonly affect the bone marrow and adjacent bone structures, with primary symptoms of bone pain and pathological fractures or lesions (osteolytic bone lesions), abnormal bleeding, anemia and increased susceptibility to infections. Advanced stages of the disease include renal failure, skeletal deformities, compaction of the spinal cord, and hypercalcemia. Myeloma affects bone cells by inducing osteoclast resorption of bone, hence decimating bone structure and increasing calcium concentration in plasma. The etiology of myelomas is currently unknown. Linkage to radiation damage, mutations in oncogenes, familial causes and abnormal IL6 expression have been postulated.

Multiple myelomas are plasma cell tumors with multiple origins. Multiple myelomas account for nearly 10% of all plasma cell malignancies, and are the most common bone tumor cancer in adults, with an annual incident rate of 3 to 4 cases per 100,000 people with a median age at diagnosis of between 63 and 70 years. In the United States, multiple myelomas are the second most common hematologic malignancy after Non-Hodgkin's Lymphoma, with approximately 50,000 cases in the United States alone, and approximately 13,500 new reported cases every year. In Europe, the incidence of multiple myelomas is 6 cases per 100,000 people per year. The prognosis outlook for patients diagnosed with multiple myelomas is grim, with only several months to a year for patients with advanced forms of the disease.

Traditional treatment regions for myeloma and multiple myelomas (henceforth referred to as “myeloma”) consist of chemotherapy, radiation therapy, and surgery. In addition, bone marrow transplantation is recommended for patients who are otherwise in good health. The cure rate for patient's approaches 30%, and is the only method known that can cure myelomas. However, for individuals who are older or cannot tolerate bone marrow transplantation procedures, chemotherapy is most appropriate.

Recently, important advances in multiple myeloma therapies such as the introduction of autologous stem cell transplantation (ASCT) and the availability of thalidomide, lenalidomide (immunomodulatory drugs or IMiDs) and bortezomib have changed the management of these patients and have allowed an increase in overall survival (OS) (Kristinsson et al., J. Clin. Oncol., 25:1993-1999 (2007); Brenner et al., Blood, 111:2521-2526 (2008); and Kumar et al., Blood, 111:2516-2520 (2008)). Patients younger than 60 years have a 10 year survival probability of ˜30% (Raab et al., Lancet, 374:324-339 (2009)). Thalidomide (Rajkumar et al., J. Clin. Oncol., 26:2171-2177 (2008)), lenalidomide (Rajkumar et al., Lancet Oncol., 11:29-37 (2010)); or bortezomib (Harousseau et al., J. Clin. Oncol., 28:4621-4629 (2010)), in combination with dexamethasone as part of an induction therapy regimen before ASCT has led to rates of nearly CR of 8, 15 and 16%, respectively; whereas three-drug induction schedules of bortezomib-dexamethasone plus doxorubicin (Sonneveld et al., Blood (ASH Annual

Meeting Abstracts), 116:23 (2010)), cyclophosphamide (Reeder et al., Leukemia, 23:1337-1341 (2009)), thalidomide (Cavo et al., Lancet, 376:2075-2085 (2010)); or lenalidomide (Richardson et al., Blood, 116:679-686 (2010)), permits achievement rates of nearly CR of 7, 39, 32 and 57%, respectively. However, despite these advances, almost all multiple myeloma patients relapse.

The appearance of abnormal antibodies, known as M-protein, is a diagnostic indicator of multiple myeloma. The increased production of M-protein has been linked to hyperviscosity syndrome in multiple myelomas, causing debilitating side effects, including fatigue, headaches, shortness of breath, mental confusion, chest pain, kidney damage and failure, vision problems and Raynaud's phenomenon (poor blood circulation, particularly fingers, toes, nose and ears). Cryoglobulinemia occurs when M-protein in the blood forms particles under cold conditions. These particles can block small blood vessels and cause pain and numbness in the toes, fingers, and other extremities during cold weather. Prognostic indicators, such as chromosomal deletions, elevated levels of beta-2 microglobulin, serum creatinine levels and IgA isotyping have also been studied. Tricot, G. et al., “Poor Prognosis in Multiple Myeloma”, Blood, 86:4250-4252 (1995).

Immunostimulatory monoclonal antibodies (mAb) represent a new and exciting strategy in cancer immunotherapy to potentiate the immune responses of the host against the malignancy (Melero et al., Nat. Rev. Cancer, 7:95-106 (2007)). Such agonistic or antagonistic mAbs bind to key receptors in cells of the immune system acting to enhance antigen presentation (e.g., anti-CD40), to provide costimulation (e.g., anti-PD1), or to counteract immunoregulation (e.g., anti-CTLA-4).

CS1 (also known as SLAMF7, CRACC, 19A, APEX-1, FOAP12, and 19A; GENBANK® Accession No. NM_021181.3, Ref. Boles et al., Immunogenetics, 52:302-307 (2001); Bouchon et al., J. Immunol., 167:5517-5521 (2001); Murphy et al., Biochem. J., 361:431-436 (2002)) is a member of the CD2 subset of the immunoglobulin superfamily. Molecules of the CD2 family are involved in a broad range of immunomodulatory functions, such as co-activation, proliferation differentiation, and adhesion of lymphocytes, as well as immunoglobulin secretion, cytokine production, and NK cell cytotoxicity. Several members of the CD2 family, such as CD2, CD58, and CD150, play a role or have been proposed to play a role in a number of autoimmune and inflammatory diseases, such as psoriasis, rheumatoid arthritis, and multiple sclerosis. It has been reported that CS1 plays a role in NK cell-mediated cytotoxicity and lymphocyte adhesion (Bouchon, A. et al., J. Immunol., 5517-5521 (2001); Murphy, J. et al., Biochem. J., 361:431-436 (2002)).

Elotuzumab is a humanized monoclonal IgG1 antibody directed against CS-1, a cell surface glycoprotein, which is highly and uniformly expressed in multiple myeloma. Elotuzumab induces significant antibody-dependent cellular cytotoxicity (ADCC) against primary multiple myeloma cells in the presence of peripheral lymphocytes (Tai et al., Blood, 112:1329-1337 (2008)). Results of three studies that evaluated the safety and efficacy of this drug administered alone (Zonder et al., Blood, 120(3):552-559 (2012)), in combination with bortezomib (Jakubowiak et al., J. Clin. Oncol., 30(16):1960-1965 (Jun. 1, 2012)), or lenalidomide and low-dose dexamethasone (Lonial et al., J. Clin. Oncol., 30:1953-1959 (2012); and Richardson et al., Blood (ASH Annual Meeting Abstracts), 116:986 (2010) for the treatment of patients with relapsed or refractory multiple myeloma, have been reported. All three combinations showed a manageable safety profile and encouraging activity. For example, a Phase I/II study evaluating the safety and efficacy of Elotuzumab in combination lenalidomide and low-dose dexamethasone for the treatment of relapsed or refractory multiple myeloma demonstrated a 33 month PFS as well as a 92% response rate for patients receiving the 10 mg/kg dose (Lonial et al., J. Clin. Oncol., 31 (2013) (Suppl., Abstr. 8542)). Phase III clinical trials of lenalidomide/dexamethasone with or without Elotuzumab in previously untreated multiple myeloma patients is ongoing, while another phase III trial designed to evaluate this same combination in the first line setting is also ongoing.

The Programmed Death 1 receptor (PD-1) is a key checkpoint receptor expressed by activated T and B cells and mediates immunosuppression. PD-1 is a member of the CD28 family of receptors, which includes CD28, CTLA-4, ICOS, PD-1, and BTLA. Two cell surface glycoprotein ligands for PD-1 have been identified, Programmed Death Ligand-1 (PD-L1) and Programmed Death Ligand-2 (PD-L2), that are expressed on antigen-presenting cells as well as many human cancers and have been shown to down-regulate T cell activation and cytokine secretion upon binding to PD-1. Inhibition of the PD-1/PD-L1 interaction mediates potent anti-tumor activity in preclinical models (U.S. Pat. Nos. 8,008,449 and 7,943,743), and the use of antibody inhibitors of the PD-1/PD-L1 interaction for treating cancer has entered clinical trials (Brahmer et al., J. Clin. Oncol., 28:3167-3175 (2010); Topalian et al., N Engl. J. Med., 366:2443-2454 (2012); Topalian et al., J. Clin. Oncol., 32(10):1020-1030 (2014); Hamid et al., N. Engl. J. Med., 369:134-144 (2013); Brahmer et al., N. Engl. J. Med., 366:2455-2465 (2012); Flies et al., Yale J. Biol. Med., 84:409-421 (2011); Pardoll, Nat. Rev. Cancer, 12:252-264 (2012); Hamid et al., Expert Opin. Biol. Ther., 13(6):847-861 (2013)).

In spite of the promising anti-tumor efficacy of several monoclonal antibodies, many tumors are refractory to treatment with a single antibody (Wilcox et al., J. Clin. Invest., 109:651-659 (2002); Verbrugge et al., Cancer Res., 72:3163-3174 (2012)), and combinations of two or more antibodies may be needed. It is thus an object of the present invention to provide improved methods for treating cancer patients with a combination of different monoclonal antibodies.

The present inventors have discovered, for the first time, that administration of a therapeutically effective amount of an anti-PD1 antibody with a therapeutically effective amount of an anti-CS1 antibody, results in synergistic regressions of multiple myeloma cells and tumors, thus establishing this combination as a potential treatment option for multiple myeloma patients.

### SUMMARY OF THE INVENTION

The present invention provides a method for treating a patient with multiple myeloma comprising the concurrent administration of a combination therapeutic regiment comprising: (i) a therapeutically effective amount of an anti-PD1 antibody; and (ii) a therapeutically effective amount of an anti-CS1 antibody, wherein said combination results in the synergistic reduction in tumor burden, tumor regression, and/or tumor development of said cancer.

The present invention provides a method for treating a patient with cancer comprising the concurrent administration of a combination therapeutic regiment comprising: (i) a therapeutically effective amount of an anti-PD1 antibody; and (ii) a therapeutically effective amount of an anti-CS1 antibody, wherein said combination results in the synergistic reduction in tumor burden, tumor regression, and/or tumor development of said cancer, wherein said cancer is selected from the group consisting of: myeloma, multiple myeloma, and smoldering myeloma, among others.

The present invention provides a method for treating a patient with multiple myeloma comprising the concurrent administration of a combination therapeutic regiment comprising: (i) a therapeutically effective amount of an anti-PD1 antibody; and (ii) a therapeutically effective amount of an anti-CS1 antibody, wherein said combination results in the synergistic reduction in tumor burden, tumor regression, and/or tumor development of said cancer, wherein said anti-CS1 antibody is Elotuzumab.

The present invention provides a method for treating a patient with cancer comprising the concurrent administration of a combination therapeutic regiment comprising: (i) a therapeutically effective amount of an anti-PD1 antibody; and (ii) a therapeutically effective amount of an anti-CS1 antibody, wherein said combination results in the synergistic reduction in tumor burden, tumor regression, and/or tumor development of said cancer, wherein said cancer is selected from the group consisting of: melanoma, multiple myeloma, smoldering myeloma, and wherein said anti-CS1 antibody is Elotuzumab.

The present invention provides a method for treating a patient with cancer comprising the concurrent administration of a combination therapeutic regiment comprising: (i) a therapeutically effective amount of an anti-PD1 antibody; and (ii) a therapeutically effective amount of an anti-CS1 antibody, wherein said combination results in the synergistic reduction in tumor burden, tumor regression, and/or tumor development of said cancer, wherein said cancer is a B-cell malignancy selected from the group consisting of: lymphoma, non-Hodgkin's lymphomas (NHL), chronic lymphocytic leukemia, follicular lymphoma, mantle cell lymphoma and diffuse large B-cell lymphoma, and wherein said anti-CS1 antibody is Elotuzumab.

The present invention provides a method for treating a patient with multiple myeloma comprising the concurrent administration of a combination therapeutic regiment comprising: (i) a therapeutically effective amount of an anti-PD1 antibody; and (ii) a therapeutically effective amount of an anti-CS1 antibody, wherein said combination results in the synergistic reduction in tumor burden, tumor regression, and/or tumor development of said cancer, and wherein said anti-PD1 antibody is nivolumab or pembrolizumab.

The present invention provides a method for treating a patient with cancer comprising the concurrent administration of a combination therapeutic regiment comprising: (i) a therapeutically effective amount of an anti-PD1 antibody; and (ii) a therapeutically effective amount of an anti-CS1 antibody, wherein said combination results in the synergistic reduction in tumor burden, tumor regression, and/or tumor development of said cancer, wherein said cancer is selected from the group consisting of: myeloma, multiple myeloma, smoldering myeloma, and wherein anti-PD1 antibody is nivolumab or pembrolizumab.

The present invention provides a method for treating a patient with multiple myeloma comprising the concurrent administration of a combination therapeutic regiment comprising: (i) a therapeutically effective amount of an anti-PD1 antibody; and (ii) a therapeutically effective amount of an anti-CS1 antibody, wherein said combination results in the synergistic reduction in tumor burden, tumor regression, and/or tumor development of said cancer, wherein said anti-CS1 antibody is Elotuzumab, and anti-PD1 antibody is nivolumab or pembrolizumab.

The present invention provides a method for treating a patient with cancer comprising the concurrent administration of a combination therapeutic regiment comprising: (i) a therapeutically effective amount of an anti-PD1 antibody; and (ii) a therapeutically effective amount of an anti-CS1 antibody, wherein said combination results in the synergistic reduction in tumor burden, tumor regression, and/or tumor development of said cancer, wherein said cancer is selected from the group consisting of: myeloma, multiple myeloma, smoldering myeloma, wherein said anti-CS1 antibody is Elotuzumab, and anti-PD1 antibody is nivolumab or pembrolizumab.

The present invention provides a method for treating a patient with cancer comprising the concurrent administration of a combination therapeutic regiment comprising: (i) a therapeutically effective amount of an anti-PD1 antibody; and (ii) a therapeutically effective amount of an anti-CS1 antibody, wherein said combination results in the synergistic reduction in tumor burden, tumor regression, and/or tumor development of said cancer, wherein said cancer is selected from the group consisting of: myeloma, multiple myeloma, smoldering myeloma, wherein said anti-CS1 antibody is Elotuzumab, anti-PD1 antibody is nivolumab or pembrolizumab, wherein said anti-PD1 antibody is administered at a dosage of about 0.03-3 mg/kg, or about 1 mg/kg, or about 3 mg/kg, or about 5 mg/kg, or about 10 mg/kg, or about 5 mg/kg, or about 10 mg/kg.

The present invention provides a method for treating a patient with cancer comprising the concurrent administration of a combination therapeutic regiment comprising: (i) a therapeutically effective amount of an anti-PD1 antibody; and (ii) a therapeutically effective amount of an anti-CS1 antibody, wherein said combination results in the synergistic reduction in tumor burden, tumor regression, and/or tumor development of said cancer, wherein said cancer is selected from the group consisting of: myeloma, multiple myeloma, smoldering myeloma, wherein said anti-CS1 antibody is Elotuzumab, anti-PD1 antibody is nivolumab or pembrolizumab, wherein anti-CS1 antibody is administered at a dosage of about 1 to 10 mg/kg, or about 20 mg/kg, once every week.

The present invention provides a method for treating a patient with cancer comprising the concurrent administration of a combination therapeutic regiment comprising: (i) a therapeutically effective amount of an anti-PD1 antibody; and (ii) a therapeutically effective amount of an anti-CS1 antibody, wherein said combination results in the synergistic reduction in tumor burden, tumor regression, and/or tumor development of said cancer, wherein said cancer is selected from the group consisting of: myeloma, multiple myeloma, smoldering myeloma, wherein said anti-CS1 antibody is Elotuzumab, anti-PD1 antibody is nivolumab or pembrolizumab, wherein SAID anti-CS1 antibody is administered at a dosage of about 1 to 10 mg/kg, or about 20 mg/kg once every 3 weeks.

The present invention provides a method for treating a patient with cancer comprising the concurrent administration of a combination therapeutic regiment comprising: (i) a therapeutically effective amount of an anti-PD1 antibody; and (ii) a therapeutically effective amount of an anti-CS1 antibody, wherein said combination results in the synergistic reduction in tumor burden, tumor regression, and/or tumor development of said cancer, wherein said cancer is selected from the group consisting of: myeloma, multiple myeloma, smoldering myeloma, wherein said anti-CS1 antibody is Elotuzumab, anti-PD1 antibody is nivolumab or pembrolizumab, wherein said anti-PD1 antibody is administered at a dosage of about 0.03-3 mg/kg, or about 1 mg/kg, or about 3 mg/kg, or about 5 mg/kg, or about 10 mg/kg, and said anti-CS1 antibody is administered at a dosage of about 1 to 10 mg/kg, or about 20 mg/kg, or about 10 mg/kg once every week, once every two weeks, or once every three weeks.

The present invention provides a method for treating a patient with cancer comprising the concurrent administration of a combination therapeutic regiment comprising: (i) a therapeutically effective amount of an anti-PD1 antibody; and (ii) a therapeutically effective amount of an anti-CS1 antibody, wherein said combination results in the synergistic reduction in tumor burden, tumor regression, and/or tumor development of said cancer, wherein said cancer is selected from the group consisting of: myeloma, multiple myeloma, smoldering myeloma, wherein said anti-CS1 antibody is Elotuzumab, anti-PD1 antibody is nivolumab or pembrolizumab, wherein said anti-PD1 antibody is administered at a dosage of about 0.03-3 mg/kg, or about 1 mg/kg, or about 3 mg/kg, or about 5 mg/kg, or about 10 mg/kg, and said anti-CS1 antibody is administered at a dosage of about 1 mg/kg once every three weeks.

The present invention provides a method for treating a patient with cancer comprising the concurrent administration of a combination therapeutic regiment comprising: (i) a therapeutically effective amount of an anti-PD1 antibody; and (ii) a therapeutically effective amount of an anti-CS1 antibody, wherein said combination results in the synergistic reduction in tumor burden, tumor regression, and/or tumor development of said cancer, wherein said cancer is selected from the group consisting of: myeloma, multiple myeloma, smoldering myeloma, wherein said anti-CS1 antibody is Elotuzumab, anti-PD1 antibody is nivolumab or pembrolizumab, wherein said anti-PD1 antibody is administered at a dosage of about 0.03-3 mg/kg, or about 1 mg/kg, or about 3 mg/kg, or about 5 mg/kg, or about 10 mg/kg, and said anti-CS1 antibody is administered at a dosage of about 10 mg/kg once every three weeks.

The present invention provides a method for treating a patient with cancer comprising the sequential administration of a combination therapeutic regiment comprising: (i) first administering a therapeutically effective amount of an anti-CS1 antibody; followed by (ii) administering a therapeutically effective amount of an anti-PD1 antibody; wherein said combination results in the synergistic reduction in tumor burden, tumor regression, and/or tumor development of said cancer, wherein said cancer is selected from the group consisting of: myeloma, multiple myeloma, smoldering myeloma, wherein said anti-PD1 antibody is nivolumab, wherein said anti-CS1 antibody is Elotuzumab, and wherein said anti-PD1 antibody is administered at a dosage of about 0.03-3 mg/kg, or about 1 mg/kg, or about 3 mg/kg, or about 5 mg/kg, or about 10 mg/kg, and said anti-CS1 antibody is administered at a dosage of about 10 mg/kg once every week, two weeks, or three weeks.

The present invention provides a method for treating a patient with cancer with a sequential administration of a combination therapeutic regiment comprising: (i) first administering a therapeutically effective amount of an anti-CS1 antibody; followed by (ii) administering a therapeutically effective amount of an anti-PD1 antibody; wherein said method optionally comprises an Intervening Period in-between (i) and (ii), wherein said Intervening Period is between 0 days to 24 weeks in time. In one aspect of the present invention, the Intervening Period is between 2 to 8 weeks. In one aspect of the present invention, the Intervening Period is between 3 to 6 weeks. In one aspect of the present invention, the Intervening Period is between 1 to 2 weeks. In one aspect of the present invention, the Intervening Period is between 3 to 7 days. In one aspect of the present invention, the Intervening Period is between about 1 to 3 days. In one aspect of the present invention, the Intervening Period is about 2 days. In one aspect of the present invention, the Intervening Period is about 1 day.

In another aspect, methods of treating multiple myeloma in a human patient are provided, the methods comprising administering to the patient, an effective amount of each of:

(a) an anti-PD1 antibody comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3, and

(b) an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1,

wherein the anti-CS1 antibody is administered weekly for a total of 8 doses over 8 weeks and the anti-PD1 antibody is administered every 3 weeks for a total of 3 doses over 8 weeks during an induction phase, and

wherein the anti-PD1 antibody is administered at a dose of about 0.03-3 mg/kg, or about 1 mg/kg, or about 3 mg/kg and the anti-CS1 antibody is administered at a dose of 10 mg/kg during both the induction and maintenance phases.

In another aspect, methods of treating multiple myeloma in a human patient are provided, the methods comprising administering to the patient, an effective amount of each of:

(a) an anti-PD1 antibody comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3, and

(b) an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1,

wherein the anti-CS1 antibody is administered weekly for a total of 8 doses over 8 weeks and the anti-PD1 antibody is administered every 3 weeks for a total of 3 doses over 8 weeks during an induction phase, and

wherein the anti-PD1 antibody is administered at a dose of 1 mg/kg and the anti-CS1 antibody is administered at a dose of 10 mg/kg body weight during both the induction and maintenance phases.

In another aspect, methods of treating multiple myeloma in a human patient are provided, the methods comprising administering to the patient, an effective amount of each of:

(a) an anti-PD1 antibody comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3, and

(b) an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1,

wherein the anti-CS1 antibody is administered weekly for a total of 8 doses over 8 weeks and the anti-PD1 antibody is administered every 3 weeks for a total of 3 doses over 8 weeks during an induction phase, and

wherein the anti-PD1 antibody is administered at a dose of 3 mg/kg and the anti-CS1 antibody is administered at a dose of 10 mg/kg body weight during both the induction and maintenance phases.

In certain embodiments, each dose of the anti-PD1 antibody is administered at about 0.3, 0.1, 0.3, 1, 3, 6, 10 or 20 mg/kg. In preferred embodiments, each dose of the anti-PD1 antibody is administered at 0.03 mg/kg, 0.1 mg/kg, 1 mg/kg or 3 mg/kg; or 3 mg or 8 mg. In other embodiments, each dose of the anti-CS1 antibody is administered at 0.1, 0.3, 1, 3, 6, 10 or 20 mg/kg body weight. In a preferred embodiment, each dose of the anti-CS1 antibody is administered at 10 mg/kg.

In one embodiment, the anti-PD1 antibody and anti-CS1 antibody are administered at the following doses during either the induction or maintenance phase:

(a) 0.03 mg/kg anti-PD1 antibody and 10 mg/kg of anti-CS1 antibody;

(b) 0.1 mg/kg anti-PD1 antibody and 10 mg/kg of anti-CS1 antibody;

(c) 0.3 mg/kg anti-PD1 antibody and 10 mg/kg of anti-CS1 antibody;

(d) 1 mg/kg anti-PD1 antibody and 10 mg/kg of anti-CS1 antibody; or

(e) 3 mg/kg anti-PD1 antibody and 10 mg/kg of anti-CS1 antibody.

In one embodiment, the anti-PD1 antibody and anti-CS1 antibody are administered at the following doses during either the induction or maintenance phase:

(a) 0.03 mg/kg anti-PD1 antibody and 1 mg/kg of anti-CS1 antibody;

(b) 0.1 mg/kg anti-PD1 antibody and 1 mg/kg of anti-CS1 antibody;

(c) 0.3 mg/kg anti-PD1 antibody and 1 mg/kg of anti-CS1 antibody;

(d) 1 mg/kg anti-PD1 antibody and 1 mg/kg of anti-CS1 antibody; or

(e) 3 mg/kg anti-PD1 antibody and 1 mg/kg of anti-CS1 antibody.

In certain embodiments, each dose of the anti-PD1 antibody is administered at about 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, or 20 mg. In preferred embodiments, each dose of the anti-PD1 antibody is administered at about 3 mg or 8 mg. In other embodiments, each dose of the anti-CS1 antibody is administered at 0.1, 0.3, 1, 3, 6, 10 or 20 mg/kg body weight.

In a preferred embodiment, each dose of the anti-CS1 antibody is administered at 10 mg/kg.

In one embodiment, the anti-CS1 antibody is administered on (1) day 1, week 1, (2) day 1, week 2, (3) day 1, week 3, (4) day 1, week 4, (5) day 1, week 5, (6) day 1, week 6, (7) day 1, week 7, and (8) day 1, week 8, of the induction phase. In another embodiment, the anti-PD1 antibody is administered on (1) day 1, week 1, (2) day 1, week 4, and (3) day 1, week 7 of the induction phase. In another embodiment, the anti-CS1 antibody is administered on (1) day 1, week 10 and (2) day 1, week 15 of the maintenance phase. In another embodiment, the anti-PD1 antibody is administered on (1) day 1, week 10 of the maintenance phase. In another embodiment, the maintenance phase is repeated for at least 1, 2, 3, 4, 5, 6, 7, 8, 9, 10, 11, 12, 13, 14, 15, 16, 17, 18, 19, or 20 or more cycles.

In one embodiment, the anti-CS1 antibody and anti-PD1 antibody are administered as a first (“front”) line of treatment (e.g., the initial or first treatment). In another embodiment, the anti-CS1 antibody and anti-PD1 antibody are administered as a second line of treatment (e.g., after initial treatment with the same or a different therapeutic, including after relapse and/or where the first treatment has failed).

The anti-PD1 antibody and anti-CS1 antibodies can be administered to a subject by any suitable means. In one embodiment, the antibodies are formulated for intravenous administration. In another embodiment, the antibodies are administered simultaneously (e.g., in a single formulation or concurrently as separate formulations). Alternatively, in another embodiment, the antibodies are administered sequentially (e.g., as separate formulations).

The efficacy of the treatment methods provided herein can be assessed using any suitable means. In one embodiment, the treatment produces at least one therapeutic effect selected from the group consisting of complete response, very good partial response, partial response, and stable disease. In another embodiment, administration of an anti-PD1 antibody and an anti-CS1 antibody has a synergistic effect on treatment compared to administration of either antibody alone.

Also provided are compositions comprising:

(a) an anti-PD1 antibody comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3, and

(b) an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1.

The invention further provides kits that include a pharmaceutical composition containing an anti-PD1 antibody, such as nivolumab or pembrolizumab, and an anti-CS1 antibody, such as Elotuzumab, and a pharmaceutically-acceptable carrier, in a therapeutically effective amount adapted for use in the methods described herein. In one embodiment, the kit comprises:

(a) a dose of an anti-PD1 antibody comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3, and

(b) a dose of an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1; and

(c) instructions for using the anti-PD1 antibody and anti-CS1 antibody in a method of the in the invention.

In another aspect, an anti-PD1 antibody is provided, the anti-PD1 antibody comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3, for co-administration with an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1.

In a further aspect, an anti-PD1 antibody is provided, the anti-PD1 antibody comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3, for co-administration with an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1,

wherein (A) the anti-CS1 antibody is administered weekly for a total of 8 doses over 8 weeks and the anti-PD1 antibody is administered every 3 weeks for a total of 3 doses over 8 weeks during an induction phase, followed by (B) administration of the anti-CS1 antibody every 2 weeks and administration of the anti-PD1 antibody every 4 weeks during a maintenance phase, and

wherein the anti-PD1 antibody is administered at a dose of 0.1-20 mg/kg body weight and the anti-CS1 antibody is administered at a dose of 0.1-20 mg/kg body weight during both the induction and maintenance phases.

In a further aspect, an anti-PD1 antibody is provided, the anti-PD1 antibody comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3, for co-administration with an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1,

wherein (A) the anti-CS1 antibody is administered weekly for a total of 8 doses over 8 weeks and the anti-PD1 antibody is administered every 3 weeks for a total of 3 doses over 8 weeks during an induction phase, followed by (B) administration of the anti-CS1 antibody every 2 weeks and administration of the anti-PD1 antibody every 4 weeks during a maintenance phase, and

wherein the anti-PD1 antibody is administered at a dose of 0.03-0.1 mg/kg body weight and the anti-CS1 antibody is administered at a dose of 0.1-20 mg/kg body weight during both the induction and maintenance phases.

In a further aspect, an anti-PD1 antibody is provided, the anti-PD1 antibody comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3, for co-administration with an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1,

wherein (A) the anti-CS1 antibody is administered weekly for a total of 8 doses over 8 weeks and the anti-PD1 antibody is administered every 3 weeks for a total of 3 doses over 8 weeks during an induction phase, followed by (B) administration of the anti-CS1 antibody every 2 weeks and administration of the anti-PD1 antibody every 4 weeks during a maintenance phase, and

wherein the anti-PD1 antibody is administered at a dose of between 3 mg-8 mg and the anti-CS1 antibody is administered at a dose of 0.1-20 mg/kg body weight during both the induction and maintenance phases.

The invention further provides kits that include a pharmaceutical composition containing an anti-PD1 antibody, such as nivolumab or pembrolizumab, and an anti-CS1 antibody, such as Elotuzumab, and a pharmaceutically-acceptable carrier, in a therapeutically effective amount adapted for use in the methods described herein. In one embodiment, the kit comprises:

(a) a dose of an anti-PD1 antibody comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3, and

(b) a dose of an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1, and

(c) instructions for first administering the anti-CS1 antibody followed by the anti-PD1 antibody thereafter.

In another aspect, an anti-PD1 antibody is provided, the anti-PD1 antibody comprising the CDR1, CDR2 and CDR3 domains in a heavy chain variable region comprising the sequence set forth in SEQ ID NO:4, and the CDR1, CDR2 and CDR3 domains in a light chain variable region comprising the sequence set forth in SEQ ID NO:3, for sequential administration with an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:1, wherein the anti-CS1 antibody is administered first followed by the anti-PD1 antibody.

### BRIEF DESCRIPTION OF THE FIGURES/DRAWINGS

FIG. 1. Amino acid sequence of human SLAMF7 (CS1-L).

FIGS. 2A-B. Murine B-Cell Lymphoma Cells (A20) stably express GFP and hSLAMF7. Cells were stained with PE-conjugated anti-human SLAMF7 (clone 162.1, BioLegend) and the frequency of cells staining positive for GFP and hSLAMF7 are shown at day 0 (A), and at day 58 (B).

FIG. 3. Elotuzumab binding to hSLAMF7 expressed in A20 cells was confirmed by flow cytometry. A20-GFP or A20-hSLAMF7-GFP cells were incubated with 6.25 ug/ml Elotuzumab (BMS), washed twice and incubated with anti-human IgG-PE secondary antibody. The frequency of cells staining positive for GFP and hSLAMF7 is shown at 0 days.

FIGS. 4A-B. A20-hSLAMF7-GFP cells grow in Balb/c mice and retain the surface expression of hSLAMF7. Tumors were established via subcutaneous injection of either 10⁷ A20-GFP or 10⁷ A20-hSLAMF7-GFP cells into the hind flank of Balb/c mice. (A) Tumor growth was measured by digital caliper twice weekly. Mice were euthanized when the tumors reached 2,000 mm³. Number of animals free of tumor by end of the experiment were designed tumor free (TF). (B) Cells isolated from A20-GFP or A20-hSLAMF7-GFP tumors were stained with anti-hSLAMF7 (clone 162.1, BioLegend) or mIgG2b isotype control antibody (MPC-11, BioLegend). Parental A20 cells maintained in culture were stained as a control. Samples were analyzed on a FACSCanto flow cytometer (BD) and percentage of cells positive for GFP and hSLAMF7 is shown.

FIGS. 5A-E. In vivo anti-tumor efficacy of Elo-mIgG2a in A20-hSLAMF7-GFP model. Mice bearing A20-hSLAMF7-GFP tumors were randomized to different treatment groups when their tumors reached an average size of 180.1±87.3 mm³. Mice bearing A20-GFP tumors had tumors with the average size of 193.3±133.2 mm³. The treatment groups consisted of treatment with Elo-mIgG2a at doses 1, 5, and 10 mg/kg. The control group received mIgG2a control antibody (Bioxcell) at 10 mg/kg. Dosing was on days 14, 17, 21, 24, and 28. Experiment was terminated on day 59. The tumor volumes of individual mice are shown for the following conditions: (A) 10 mg/kg Elotuzumab-mIgG2a for mice bearing A20-GFP tumors; (B) 10 mg/kg mIgG2b isotype control antibody for mice bearing A20-SLAMF7-GFP tumors; (C) 1 mg/kg Elotuzumab-mIgG2a for mice bearing A20-SLAMF7-GFP tumors; (D) 5 mg/kg Elotuzumab-mIgG2a for mice bearing A20-SLAMF7-GFP tumors; and (E) 10 mg/kg Elotuzumab-mIgG2a for mice bearing A20-SLAMF7-GFP tumors.

FIGS. 6A-B. (A) Mean and (B) median tumor volumes across five treatment groups are shown for mice bearing A20-hSLAMF7-GFP tumors.

FIG. 7. Tumor growth delay (TGD) for different treatment groups related to the isotype control antibody (Iso 10 mg/kg) calculated at 4 predetermined tumor volumes using Elo-mIgG2a (“Elo-g2a”) at 3 different doses. TGD was calculated using mice treated with 1 mg/kg (n=6), 5 mg/kg (n=8) and 10 mg/kg (n=8) Elo-mIgG2a. In view of these results, 10 mg/kg of Elo-mIgG2a was selected for combination experiments with anti-PD1.

FIG. 8. Elo-mIgG2a concentrations in tumor bearing Balb/c mice treated with varying doses of Elo-mIgG2a (“Elo”). Blood samples were collected at various time points from tumor bearing mice described in FIG. 5. Blood was collected prior to treatment (pre-bleed, day 14), at 8 hours after the first dose (day 15), immediately before the second dose (day 17), immediately before the last dose (day 28), and 8 hours after the last dose (day 29). N=3-9 mice/group. Sera were then analyzed by Enzyme-linked Immunosorbent Assay (ELISA). Serum samples were diluted 64,000-fold. Anti-idiotype monoclonal antibody to Elotuzumab (BMS) was used to capture Elo-mIgG2a in mouse serum samples. The captured Elo-mIgG2a was detected using anti-mouse IgG2a-HRP and measured using TMB substrate. The results showed that maximal anti-tumor activity correlated with 110±49 μg/mL (before the second dose)-357±111 μg/mL (after the last dose) for the 10 mg/kg dose of Elo-mIgG2a while lower biological activity correlated with levels of 5±2-27±7 μg/mL for the 1 mg/kg dose of Elo-mIgG2a. Serum levels of Elo-mIgG2a were similar in mice bearing A20-hSLAMF7-GFP and A20-GFP tumors (110±49-357±111 μg/mL vs. 102±30-381±43 μg/mL) for the 10 mg/kg dose of Elo-mIgG2a.

FIG. 9. PD-L1 is expressed on parental A20, A20-GFP, and A20-hSLAMF7-GFP cell lines. Flow cytometric analysis of PDL1 expression is shown. Cells were unstained (light grey line within first peak in histogram) or stained with either rat IgG2b (RTK4530, BioLegend) (dark grey, outer first peak in histogram) or rat anti-mouse PD-L1 (10F.9G2, BioLegend) (second peak in histogram).

FIGS. 10A-F. Anti-PD-1 significantly enhanced Elo-mIgG2a-mediated anti-tumor activity in A20-hSLAMF7-GFP mice in vivo relative to either Elo-mIgG2a or anti-PD-1 as single agents. The treatment groups consisted of treatment with (A) isotype controls mIgG2a at 10 mg/kg and mIgG1 at 10 mg/kg; (B) isotype control mIgG2a in combination with anti-PD-1 at 3 mg/kg; (C) isotype control mIgG2a in combination with anti-PD-1 at 1 mg/kg; (D) isotype control mIgG1 in combination with Elo-mIgG2 at 10 mg/kg; (E) Elo-mIgG2 at 10 mg/kg in combination with anti-PD-1 at 3 mg/kg; and (F) Elo-mIgG2 at 10 mg/kg in combination with anti-PD-1 at 1 mg/kg. Elo-mIgG2a/mIgG2a was administered on days 10, 14, 17, 21 and 24 (5 doses). Anti-PD-1 or mIgG1 was administered on days 10, 14 and 17 (3 doses). (i) indicates when anti-PD1 treatment ended, while (ii) indicates when Elo-mIgG2 treatment ended. Experiment was terminated on day 44. Tumor volumes were measured biweekly. The number of tumor-free (TF) mice per group is shown for each group. As shown, A20-hSLAMF7-GFP mice treated with Elo-mIgG2 at 10 mg/kg in combination with anti-PD-1 at 3 mg/kg resulted in the synergistic reduction in tumor burden as evidenced by 8 out of 9 mice being designated as tumor free, compared to only 2 out of 9 mice with either agent alone.

FIGS. 11A-B. Comparison of the different treated mouse groups at day 21 post tumor engraftment. (A) Data are expressed as individual tumor volume and median for each of treatments tested using either control antibodies (“mIgG2a” or “mIgG1”), Elo-mIgG2 antibody (“Elo-g2a”), or the anti-mouse PD1 antibody (“PD1”), and combinations thereof (B) Statistical analysis: all groups were compared with a Kruskal-Wallis non parametric test followed by a Dunn's multiple comparison test. P values are shown.

FIGS. 12A-F. Anti-tumor activity of Elo-g2a antibody, anti-PD1 antibody, or their combination in A20-hSLAMF7-GFP tumor model following different schedules of administration. Concurrent administration of anti-PD-1 antibody and Elo-mIgG2a antibody significantly enhances anti-tumor activity in A20-hSLAMF7-GFP mice in vivo relative to sequential administration. The treatment groups consisted of treatment with (A) isotype controls mIgG2a at 10 mg/kg and mIgG1 at 10 mg/kg were administered on days 11, 14, and 18; (B) anti-PD-1 at 3 mg/kg on days 11, 14, and 18; (C) Elo-mIgG2 at 10 mg/kg on days 11, 14, and 18; (D) Concurrent administration of Elo-mIgG2 at 10 mg/kg and anti-PD-1 at 3 mg/kg on days 11, 14, and 18; (E) Sequential administration of Elo-mIgG2 at 10 mg/kg on day 11, followed by the combination of anti-PD-1 at 3 mg/kg and Elo-g2a at 10 mg/kg on days 14 and 18; and (F) Sequential administration of Elo-mIgG2 at 10 mg/kg on day 11, followed by anti-PD-1 at 3mg/kg on days 14 and 18. The vertical dotted line when treatment ended. Experiment was terminated on day 40. Tumor volumes were measured biweekly. The number of tumor-free (TF) mice per group is shown for each group. As shown, concurrent administration of Elo-mIgG2 and anti-PD-1 resulted in significant improvement in the anti-tumor effects compared to sequential treatment.

FIG. 13. Binary logistic regression analysis of tumor free mice in four independent studies 21 days post treatment with either control antibodies (“mIgG2a” or “mIgG1”), Elo-mIgG2 (“Elo-g2a”), or the anti-mouse PD1 antibody (“PD1”), and combinations thereof. N=5-12 mice/group per study. Significance is denoted as ** with p<0.01; and *** p<0.0001.

FIGS. 14A-D. Anti-tumor activity of Elo-g2a antibody, anti-PD1 antibody, or their combination in EG7-hSLAMF7-GFP tumor model. The treatment groups consisted of treatment with (A) Isotype controls; (B) anti-PD-1, 10 mg/kg; (C) Elo-g2a, 10 mg/kg; and (D) anti-PD-1, 10 mg/kg +Elo-g2a, 10 mg/kg (concurrent treatment). Dosing was performed on days 7, 10, and 14. The experiment was terminated on day 28. Tumor volumes were measured biweekly. The number of tumor-free (TF) mice per group is shown for each group. As shown, concurrent administration of Elo-mIgG2 and anti-PD-1 in the EG7 mouse tumor model resulted in significant improvement in the anti-tumor effects compared to sequential treatment.

### DETAILED DESCRIPTION OF THE INVENTION

The present invention is based on data from preclinical studies conducted in Balb/c mice (8-10 weeks old) that were implanted SC (subcutaneous implantation) with A20-hSLAMF7-GFP tumors which were treated via IP (intraperitoneal administration) with a form of Elotuzumab that was modified to contain murine IgG2 (referred to as Elo-mIgG2a), or treated with an anti-mouse PD1 mAb (4H2) alone or in combination with each other. The results demonstrated for the first time that the combination of Elotuzumab and an anti-PD1 mAb resulted in a synergistic number of mice exhibiting complete, tumor free, responses compared to either Elotuzumab or anti-CD1 mAb alone. In particular, when anti-PD1 mAb and Elotuzumab were administered, complete regressions were observed in 8 out of 9 mice when the anti-PD1 mAb was administered at 3 mg/kg, compared to only 2 out of 9 mice for either anti-PD1 or Elotuzumab alone. In addition, enhanced tumor free responses were observed when the anti-PD1 mAb was administered at a dose of 1 mg/kg in combination with Elotuzumab.

On account of the A20 cell line representing a murine B-cell lymphoma cell line, the results also demonstrate the utility of treating B-cell lymphomas and other B-cell malignancies with Elotuzumab in combination with an anti-PD1 antibody.

The teachings of the present invention are believed to be the first association between the administration of an anti-CS1 agent in combination with an anti-PD1 agent with increased, and in some cases synergistic, outcomes in terms of efficacy, safety, and tolerability.

The teachings of the present invention are believed to be the first association between the administration of an anti-CS1 agent in combination with an anti-PD1 agent with increased, and in some cases synergistic outcomes, particularly when the anti-CS1 agent is administered at a dose of about 10 mg/kg, and the anti-PD1 agent is administered at a dose between about 1 to 3 mg/kg.

For the purposes of the present invention, an anti-CS1 agent may be administered either concurrently or sequentially with an anti-PD1 agent.

Concurrent administration is intended to mean an the anti-CS1 agent and anti-PD1 agent are administered at the same time, at essentially the same time, at about the same time, or within a reasonable period of time of a few minutes, to a few hours, or even as long as one or two days apart from each other.

The phrase “sequential dosing regimen”, generally refers to treating a patient with at least two agents in a specific order, wherein one cycle of a first agent is administered after the cycle of other agent (e.g., anti-CS1 agent is administered first followed by the administration of an anti-PD1 agent, or anti-PD1 agent is administered first followed by the administration of an anti-CS1 agent). In addition, the phrase “sequential dosing regimen” also encompasses the phrase “phased dosing regimen” as it is traditionally referred to in the pharmaceutical arts. In one context, “sequential dosing regimen” refers to not only the order in which the cycles are administered, but also to the entire treatment regimen for the patient. For example, “sequential dosing regimen” may include the complete dosing regimen for the patient including one or more cycles of an anti-CS1 agent, followed by one or more cycles of either an anti-PD1 agent or a combination comprising an anti-PD1 agent and one or more anti-CS1 agent. In one embodiment, the anti-CS1 or anti-PD1 agent may be administered about 1, about 2, about 3, about 4, about 5, about 6, about 7, about 8, about 9, about 10, about 11, about 12, about 13, or about 14 days after either the anti-CS1 or anti-PD1 agent is administered. In another embodiment, the anti-CS1 or anti-PD1 agent may be administered about 1, about 2, about 3, about 4, about 5, about 6, about 7, about 8, about 9, about 10, about 11, about 12, about 13, or about 14 weeks after either the anti-CS1 or anti-PD1 agent is administered. In this context, the term “about” shall be construed to mean±1, 2, 3, 4, 5, 6, or 7 days more or less than the stated period.

The concurrent administration of an anti-CS1 agent with an anti-PD1 agent, or the sequential administration of an anti-CS1 agent followed by an anti-PD1 agent, may be administered after a sufficient period of time after a patients prior therapy has passed, which may be at least about 3 weeks, about 4 weeks, about 5 weeks, about 6 weeks, about 7 weeks, about 8 weeks, about 9 weeks, about 10 weeks, about 11 weeks, about 12 weeks, or more weeks after the patients prior therapy has ended and/or after the physician has determined the prior therapy had failed.

In one aspect of the present invention, the sequential administration of one or more cycles of an anti-CS1 agent followed by one or more cycles comprising an anti-PD1 agent, may optionally comprise an “Intervening Period”, defined as a time period beginning from the end of the last anti-CS1 agent cycle up until the beginning of the anti-PD1 agent cycle. In another aspect of the present invention, the sequential administration of one or more cycles of an anti-PD1 agent followed by one or more cycles comprising an anti-CS1 agent, may optionally comprise an “Intervening Period”, defined as a time period beginning from the end of the last anti-CS1 agent cycle up until the beginning of the anti-PD1 agent cycle. The Intervening Period may be about 24 weeks. In another embodiment of the present invention, the Intervening Period may be about 20 weeks. In another embodiment of the present invention, the Intervening Period may be about 18 weeks. In another embodiment of the present invention, the Intervening Period may be about 15 weeks. In another embodiment of the present invention, the Intervening Period may be about 12 weeks. In another embodiment of the present invention, the Intervening Period may be about 11 weeks. In another embodiment of the present invention, the Intervening Period may be about 10 weeks. In another embodiment of the present invention, the Intervening Period may be about 9 weeks. In another embodiment of the present invention, the Intervening Period may be about 8 weeks. In another embodiment of the present invention, the Intervening Period may be about 7 weeks. In another embodiment of the present invention, the Intervening Period may be about 6 weeks. In another embodiment of the present invention, the Intervening Period may be about 5 weeks. In another embodiment of the present invention, the Intervening Period may be about 4 weeks. In another embodiment of the present invention, the Intervening Period may be about 3 weeks. In another embodiment of the present invention, the Intervening Period may be about 2 weeks. In another embodiment of the present invention, the Intervening Period may be about 1 week. In another embodiment of the present invention, the Intervening Period may be about 1, 2, 3, 4, 5, 6, or 7 days. In this context, the term “about” shall be construed to mean ±1, 2, 3, 4, 5, 6, or 7 days more or less than the stated Intervening Period.

In one embodiment of the present invention, the Intervening Period is between 2 to 8 weeks. In another embodiment of the present invention, the Intervening Period is between 3 to 6 weeks.

In one embodiment of the present invention, the Intervening Period is one day.

In another embodiment of the present invention, the Intervening Period may be less than 0 days such that the anti-CS1 agent is administered concurrently with the anti-PD1 agent.

The phrase “an anti-PD1 cycle” or “cycle of an anti-PD1 agent” is meant to encompass either one or more dosing cycle(s) of an anti-PD1 agent, or one or more dosing cycle(s) of a combination comprising one or more anti-PD1 agent(s).

The phrase “an anti-CS1 cycle” or “cycle of an anti-CS1 agent” or “cycles of a therapeutically effective amount of an anti-CS1 antibody” is meant to encompass either one or more dosing cycle(s) of an anti-CS1 agent, or one or more dosing cycle(s) of a combination comprising one or more anti-CS1 agent(s).

For the purposes of the present invention, “one or more cycles of an anti-PD1 agent cycle” and/or “one or more cycles of an anti-PD1 agent” means at least 1, at least 2, at least 3, at least 4, at least 5, at least 6, at least 7, at least 8, at least 9, or at least 10 cycles of primary treatment with either agent(s), followed by one or more optional maintenance cycles of either agent(s). The maintenance cycle(s) may follow a similar number of cycles as outlined for the primary therapy, or may be significantly longer or shorter in terms of cycle number, depending upon the patient's disease and/or severity.

For the purposes of the present invention, “one or more cycles of an anti-CS1 cycle” and/or “one or more cycles of an anti-CS1 agent” means at least 1, at least 2, at least 3, at least 4, at least 5, at least 6, at least 7, at least 8, at least 9, or at least 10 cycles of primary treatment with either agent(s), followed by one or more optional maintenance cycles of either agent(s). The maintenance cycle(s) may follow a similar number of cycles as outlined for the primary therapy, or may be significantly longer or shorter in terms of cycle number, depending upon the patient's disease and/or severity.

In another aspect of the present invention, the sequential dosing regimen may comprise a “hybrid cycle” in which the patient is administered one or more anti-CS1 agent cycles, followed by one or more anti-PD1 agent cycles, followed by one or more anti-CS1 agent cycles and/or one or more anti-PD1 agent cycles, and vice versa.

The phrase “clinical benefit” or “benefit” refers to a condition where a patient achieves a complete response; partial response; stable disease; or as otherwise described herein.

In another aspect of the present invention, the concurrent administration of an anti-CS1 agent with an anti-PD1 agent, or the sequential administration of an anti-CS1 agent followed by an anti-PD1 agent, may be administered in further combination with one or more immunomodulatory agents, co-stimulatory pathway modulators.

The phrase “anti-CS1 agent” generally refers to an agent that binds to CS1, may modulate and/or inhibit CS1 activity, may activate NK cells, and may be an anti-CS1 antibody, including Elotuzumab.

The phrase “anti-PD1 agent” generally refers to an agent that binds to PD1, may modulate and/or inhibit PD1 activity, may inhibit one of its ligands (PDL1, PDL2, etc.) to bind to the PD1 receptor, and may be an anti-PD1 antibody, including nivolumab and pembrolizumab.

The phrase “immunomodulatory agent” generally refers to an agent that either increases or decreases the function of the immune system, and/or as defined elsewhere herein, and includes co-stimulatory pathway modulators, Ipilimumab; ORENCIA®; Belatacept; CD28 antagonists, CD80 antagonists, CD86 antagonists, PD1 antagonists, PDL1 antagonists, CTLA-4 antagonists, and KIR antagonists, among others disclosed herein.

The phrase “co-stimulatory pathway modulator”, generally refers to an agent that functions by increasing or decreasing the function of the immune system by modulating the co-stimulatory pathway. In one aspect of the present invention, a co-stimulatory pathway modulator is an immunostimulant or T-cell activator, and may also encompass any agent that is capable of disrupting the ability of CD28 antigen to bind to its cognate ligand, to inhibit the ability of CTLA-4 to bind to its cognate ligand, to augment T cell responses via the co-stimulatory pathway, to disrupt the ability of B7 to bind to CD28 and/or CTLA-4, to disrupt the ability of B7 to activate the co-stimulatory pathway, to disrupt the ability of CD80 to bind to CD28 and/or CTLA-4, to disrupt the ability of CD80 to activate the co-stimulatory pathway, to disrupt the ability of CD86 to bind to CD28 and/or CTLA-4, to disrupt the ability of CD86 to activate the co-stimulatory pathway, and to disrupt the co-stimulatory pathway, in general from being activated. This necessarily includes small molecule inhibitors of CD28, CD80, CD86, CTLA-4, among other members of the co-stimulatory pathway; antibodies directed to CD28, CD80, CD86, CTLA-4, among other members of the co-stimulatory pathway; antisense molecules directed against CD28, CD80, CD86, CTLA-4, among other members of the co-stimulatory pathway; adnectins directed against CD28, CD80, CD86, CTLA-4, among other members of the co-stimulatory pathway, RNAi inhibitors (both single and double stranded) of CD28, CD80, CD86, CTLA-4, among other members of the co-stimulatory pathway, among other anti-CTLA-4 antagonists.

Anti-CTLA-4 antagonist agents for use in the methods of the invention, include, without limitation, anti-CTLA-4 antibodies, human anti-CTLA-4 antibodies, mouse anti-CTLA-4 antibodies, mammalian anti-CTLA-4 antibodies, humanized anti-CTLA-4 antibodies, monoclonal anti-CTLA-4 antibodies, polyclonal anti-CTLA-4 antibodies, chimeric anti-CTLA-4 antibodies, MDX-010 (Ipilimumab), tremelimumab, anti-CD28 antibodies, anti-CTLA-4 adnectins, anti-CTLA-4 domain antibodies, single chain anti-CTLA-4 fragments, heavy chain anti-CTLA-4 fragments, light chain anti-CTLA-4 fragments, modulators of the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP 1212422 B1. Additional CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. 2002/0039581 and 2002/086014. Other anti-CTLA-4 antibodies that can be used in a method of the present invention include, for example, those disclosed in: PCT Publication No. WO 98/42752; U.S. Pat. Nos. 6,682,736 and 6,207,156; Hurwitz et al., Proc. Natl. Acad. Sci. USA, 95(17):10067-10071 (1998); Camacho et al., J. Clin. Oncology, 22(145):Abstract No. 2505 (2004) (antibody CP-675206); Mokyr et al., Cancer Res., 58:5301-5304 (1998), and U.S. Pat. Nos. 5,977,318, 6,682,736, 7,109,003, and 7,132,281. Each of these references is specifically incorporated herein by reference for purposes of description of CTLA-4 antibodies. A preferred clinical CTLA-4 antibody is human monoclonal antibody 10D1 (also referred to as MDX-010 and Ipilimumab and available from Medarex, Inc., Bloomsbury, N.J.), disclosed in PCT Publication No. WO 01/14424.

As is known in the art, Elotuzumab refers to an anti-CS1 antibody, and is a humanized antibody anti-CS1 monoclonal antibody that enhances natural killer cell mediated antibody dependent cellular cytotoxicity of CS1 expressing myeloma cells. Elotuzumab can also be referred to as BMS-901608, or by its CAS Registry No. 915296-00-3, and is disclosed as antibody HuLuc63 in PCT Publication No. WO 2004/100898, incorporated herein by reference in its entirety and for all purposes. Specifically, Elotuzumab describes a humanized monoclonal antibody or antigen-binding portion thereof that specifically binds to CS-1, comprising a light chain variable region and a heavy chain variable region having a light chain variable region comprised of SEQ ID NO:1, and comprising a heavy chain region comprised of SEQ ID NO:2, or antigen binding fragments and variants thereof. Elotuzumab may also be described as an antibody comprising a heavy chain CDR1 having amino acids 31-35 of SEQ ID NO:2: a heavy chain CDR2 having amino acids 50-66 of SEQ ID NO:2; and a heavy chain CDR3 having amino acids 99-108 of SEQ ID NO:2; in addition to a light chain CDR1 having amino acids 24-34 of SEQ ID NO:1; a light chain CDR2 having amino acids 50-56 of SEQ ID NO:1; and a light chain CDR3 having amino acids 89-97 of SEQ ID NO: 1. Pharmaceutical compositions of Elotuzumab include all pharmaceutically acceptable compositions comprising Elotuzumab and one or more diluents, vehicles and/or excipients. Elotuzumab may be administered by I.V. at a dose of about 1 mg/kg, 10 mg/kg, about 20 mg/kg, or between about 10 to about 20 mg/kg.

Light chain variable region for Elotuzumab:
(SEQ ID NO: 1)
DIQMTQSPSSLSASVGDRVTITCKASQDVGIAVAWYQQKPGKVPKWYWAS
TRHTGVPDRFSGSGSGTDFTLTISSLQPEDVATYYCQQYSSYPYTFGQGT
KVEIK
Heavy chain variable region for Elotuzumab:
(SEQ ID NO: 2)
EVQLVESGGGLVQPGGSLRLSCAASGFDFSRYWMSWVRQAPGKGLEWIGE
INPDSSTINYAPSLKDKFIISRDNAKNSLYLQMNSLRAEDTAVYYCARPD
GNYWYFDVWGQGTLVTVSS

As is known in the art, Nivolumab refers to an anti-PD1 antibody, and is a fully human IgG4 antibody derived from transgenic mice having human genes encoding heavy and light chains to generate a functional human repertoire. Nivolumab is also referred to as BMS-936558, MDX-1106 ONO-4538, or by its CAS Registry No. 946414-94-4, and is disclosed as antibody 5C4 in WO 2006/121168, incorporated herein by reference in its entirety and for all purposes. Specifically, BMS-936558 describes a human monoclonal antibody or antigen-binding portion thereof that specifically binds to PD1, comprising a light chain variable region provided as SEQ ID NO:3, and a heavy chain variable region provided as SEQ ID NO:4, or antigen binding fragments and variants thereof. Nivolumab may also be described as an antibody comprising a light chain CDR1 having amino acids 24-34 of SEQ ID NO:3, a light chain CDR2 having amino acids 50-56 of SEQ ID NO:3, and a light chain CDR3 having amino acids 89-97 of SEQ ID NO:3; and comprising a heavy chain CDR1 having amino acids 31-35 of SEQ ID NO:4, a heavy chain CDR2 having amino acids 50-66 of SEQ ID NO:4, and a heavy chain CDR3 having amino acids 99-102 of SEQ ID NO:4. Pharmaceutical compositions of BMS-936558 include all pharmaceutically acceptable compositions comprising BMS-936558 and one or more diluents, vehicles and/or excipients. BMS-936558 may be administered by I.V.

Light chain variable region for Nivolumab:
(SEQ ID NO: 3)
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIK
Heavy chain variable region for Nivolumab:
(SEQ ID NO: 4)
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSS

As noted elsewhere herein, the administration of an anti-CS1 agent and/or an ani-PD1 agent, may be administered either alone or in combination with a peptide antigen (e.g., gp100). A non-limiting example of a peptide antigen would be a gp100 peptide comprising, or alternatively consisting of, the sequence selected from the group consisting of: IMDQVPFSV (SEQ ID NO:5), and YLEPGPVTV (SEQ ID NO:6). Such a peptide may be administered orally, or preferably at 1 mg emulsified in incomplete Freund's adjuvant (IFA) injected s.c. in one extremity, and 1 mg of either the same or a different peptide emulsified in IFA may be injected in another extremity.

Disorders for which the concurrent and/or sequential dosing regimens of the present invention may be useful in treating include, but are not limited to: multiple myeloma, melanoma, primary melanoma, unresectable stage III or IV malignant melanoma, lung cancer, non-small cell lung cancer, small cell lung cancer, prostate cancer; solid tumors, pancreatic cancer, prostatic neoplasms, breast cancer, neuroblastoma, kidney cancer, ovarian cancer, sarcoma, bone cancer, testicular cancer, hematopoietic cancers, leukemia, lymphoma, multiple myeloma, and myelodysplastic syndromes.

Additional disorders for which the concurrent and/or sequential dosing of the present invention may be useful in treating include, but are not limited to the following: glioma, gastrointestinal cancer, renal cancer, ovarian cancer, liver cancer, colorectal cancer, endometrial cancer, kidney cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, bone cancer, bone tumors, adult malignant fibrous histiocytoma of bone; childhood malignant fibrous histiocytoma of bone, sarcoma, pediatric sarcoma, sinonasal natural killer, neoplasms, plasma cell neoplasm; myelodysplastic syndromes; neuroblastoma; testicular germ cell tumor, intraocular melanoma, myelodysplastic syndromes; myelodysplastic/myeloproliferative diseases, synovial sarcoma, chronic myeloid leukemia, acute lymphoblastic leukemia, Philadelphia chromosome positive acute lymphoblastic leukemia (Ph+ ALL), multiple myeloma, acute myelogenous leukemia, chronic lymphocytic leukemia, mastocytosis and any symptom associated with mastocytosis, and any metastasis thereof. In addition, disorders include urticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, mast cell leukemia, in addition to other cancers. Other cancers are also included within the scope of disorders including, but are not limited to, the following: carcinoma, including that of the bladder, urothelial carcinoma, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burkitt's lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma, and any metastasis thereof.

The terms “treating”, “treatment” and “therapy” as used herein refer to curative therapy, prophylactic therapy, preventative therapy, and mitigating disease therapy.

The phrase “more aggressive dosing regimen” or “increased dosing frequency regimen”, as used herein refers to a dosing regimen that necessarily exceeds the basal and/or prescribed dosing regimen of either the anti-CS1 agent arm of the dosing regimen and/or the anti-PD1 arm of the dosing regimen, either due to an increased dosing frequency (about once a week, about biweekly, about once daily, about twice daily, etc.), increased or escalated dose (in the case of the anti-CS1 antibody: about 10, about 11, about 12, about 13, about 14, about 15, about 16, about 17, about 18, about 19, about 20, about 21, about 22, about 23, about 24, about 25, about 26, about 27, about 28, about 29, about 30, about 35, about 40 mg/kg; or in the case of the anti-PD1 antibody: about 0.01 mg/kg, about 0.02 mg/kg, about 0.03 mg/kg, about 0.05 mg/kg, about 0.075 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.2 mg/kg, about 1.4 mg/kg, about 1.6 mg/kg, about 1.8 mg/kg, or about 2.0 mg/kg; or about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, or about 16 mg), or by changing the route of administration which may result in an increased, bio-available level of said anti-CS1 agent and/or said the anti-PD1 agent.

In certain embodiments, the anti-PD-1 antibody is administered at a dose ranging from about 0.1 to 10.0 mg/kg body weight once every 1, 2, 3 or 4 weeks. For example, the anti-PD-1 antibody is administered at a dose of 1 or 3 mg/kg body weight once every 2 weeks.

It is to be understood this invention is not limited to particular methods, reagents, compounds, compositions, or biological systems, which can, of course, vary. It is also to be understood that the terminology used herein is for the purpose of describing particular aspects only, and is not intended to be limiting.

As used in this specification and the appended claims, the singular forms “a”, “an”, and “the” include plural referents unless the content clearly dictates otherwise. Thus, for example, reference to “a peptide” includes a combination of two or more peptides, and the like.

“About” as used herein when referring to a measurable value such as an amount, a temporal duration, and the like, is meant to encompass variations of ±20% or ±10%, preferably ±5%, or ±1%, or as little as ±0.1% from the specified value, as such variations are appropriate to perform the disclosed methods, unless otherwise specified herein.

As used herein, the terms CS1, SLAMF7, SLAM Family Member 7, CD2 Subset, CRACC, CD2-Like Receptor-Activating Cytotoxic Cells, 19A24 Protein, 19A, CD2-Like Receptor Activating Cytotoxic Cells, CD319, Novel LY9 (Lymphocyte Antigen 9) Like Protein, Membrane Protein FOAP-12, CD319 Antigen, Protein 19A, APEX-1, FOAP12, and Novel Ly93 are used interchangeably, and include variants, isoforms, species homologs of human CS1, and analogs having at least one common epitope with CS1.

CS1 is a cell surface glycoprotein that is highly expressed on Multiple Myeloma cells. CS1 is characterized by two extracellular immunoglobulin (Ig)-like domains and an intracellular signaling domain with immune receptor tyrosine-based switch motifs (Tai, Y.-T. et al., Blood, 113(18):4309-4318 (Apr. 30, 2009); Bhat, R. et al., J Leukoc. Biol., 79:417-424 (2006); Fischer, A. et al., Curr. Opin. Immunol., 19:348-353 (2007); Boles, K. S. et al., Immunogenetics, 52:302-307 (2001); Lee, J. K. et al., J. Immunol., 179:4672-4678 (2007); and Veillette, A., Immunol. Rev., 214:22-34 (2006)). CS1 is expressed at high levels in normal and malignant plasma cells, but not normal organs, solid tumors, or CD34⁺ stem cells. Only a small subset of resting lymphocytes, including NK cells and a subset of CD8⁺ T cells, express detectable but low levels of CS1_(His, E. D. et al., Clin. Cancer Res., 14:2775-2784 (2008) and Murphy, J. J. et al., Biochem. J., 361:431-436 (2002)).

CS1 (SLAMF7) was isolated and cloned by Boles et al. (Immunogenetics, 52(3-4):302-307 (2001)). The complete CS1 sequence can be found under GENBANK® Accession No. NM_021181.3 and is as follows:

(SEQ ID NO: 7)
MAGSPTCLTLIYILWQLTGSAASGPVKELVGSVGGAVTFPLKSKVKQVDS
IVWTFNTTPLVTIQPEGGTIIVTQNRNRERVDFPDGGYSLKLSKLKKNDS
GIYYVGIYSSSLQQPSTQEYVLHVYEHLSKPKVTMGLQSNKNGTCVTNLT
CCMEHGEEDVIYTWKALGQAANESHNGSILPISWRWGESDMTFICVARNP
VSRNFSSPILARKLCEGAADDPDSSMVLLCLLLVPLLLSLFVLGLFLWFL
KRERQEEYIEEKKRVDICRETPNICPHSGENTEYDTIPHTNRTILKEDPA
NTVYSTVEIPKKMENPHSLLTMPDTPRLFAYENVI

As used herein, the terms PD1, PD-1, hPD-1, CD279, SLEB2; hSLE1, and PDCD1 and Programmed Death-1, are used interchangeably, and include variants, isoforms, species homologs of human PD1, and analogs having at least one common epitope with PD1.

“Programmed Death-1 (PD-1)” refers to an immunoinhibitory receptor belonging to the CD28 family. PD-1 is expressed predominantly on previously 15 activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GENBANK® Accession No. U64863.

The complete human PD1 sequence can be found under GENBANK® Accession No. U64863 and is as follows:

(SEQ ID NO: 8)
MQIPQAPWPVVWAVLQLGWRPGWFLDSPDRPWNPPTFFPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKAQIKESLRAELRVTERRAE
VPTAHPSPSPRPAGQFQTLVVGVVGGLLGSLVLLVWVLAVICSRAARGTI
GARRTGQPLKEDPSAVPVFSVDYGELDFQWREKTPEPPVPCVPEQTEYAT
IVFPSGMGTSSPARRGSADGPRSAQPLRPEDGHCSWPL

Specific concurrent and/or sequential dosing regimens for any given patient may be established based upon the specific disease for which the patient has been diagnosed, or in conjunction with the stage of the patient's disease. For example, if a patient is diagnosed with a less-aggressive cancer, or a cancer that is in its early stages, the patient may have an increased likelihood of achieving a clinical benefit and/or immune-related response to a concurrent administration of an anti-CS1 agent followed by an anti-PD1 agent and/or a sequential administration of an anti-CS1 agent followed by an anti-PD1 agent. Alternatively, if a patient is diagnosed with a more-aggressive cancer, or a cancer that is in its later stages, the patient may have a decreased likelihood of achieving a clinical benefit and/or immune-related response to said concurrent and/or sequential administration, and thus may suggest that either higher doses of said anti-CS1 agent and/or said anti-PD1 agent therapy should be administered or more aggressive dosing regimens or either agent or combination therapy may be warranted. In one aspect, an increased dosing level of an anti-CS1, such as Ipilimumab, would be about 10, 20, 30, 40, 50, 60, 70, 80, 90, or 95% more than the typical anti-CS1 agent dose for a particular indication or individual (e.g., about 0.3 mg/kg, about 1 mg/kg, about 3 mg/kg, about 10 mg/kg, about 15 mg/kg, about 20 mg/kg, about 25 mg/kg, about 30 mg/kg), or about 1.5×, 2×, 2.5×, 3×, 3.5×, 4×, 4.5×, 5×, 6×, 7×, 8×, 9×, or 10× more anti-CS1 agent than the typical dose for a particular indication or for individual. In another aspect, an increased dosing level of an anti-PD1 agent would be about 10, 20, 30, 40, 50, 60, 70, 80, 90, or 95% more than the typical anti-PD1 agent dose for a particular indication or individual (e.g., about 0.03 mg/kg, 0.1 mg/kg, 0.3 mg/kg, about 3 mg/kg, about 10 mg/kg, about 15 mg/kg, about 20 mg/kg, about 25 mg/kg, about 30 mg/kg; or about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg or about 16 mg), or about 1.5×, 2×, 2.5×, 3×, 3.5×, 4×, 4.5×, 5×, 6×, 7×, 8×, 9×, or 10× more anti-PD1 agent than the typical dose for a particular indication or for individual.

A therapeutically effective amount of an anti-CS1 agent and/or an anti-PD1 agent, can be orally administered if it is a small molecule modulator, for example, or preferably injected into the patient, for example if it is a biologic agent. The actual dosage employed can be varied depending upon the requirements of the patient and the severity of the condition being treated. Determination of the proper starting dosage for a particular situation is within the skill of the art, though the assignment of a treatment regimen will benefit from taking into consideration the indication and the stage of the disease. Nonetheless, it will be understood that the specific dose level and frequency of dosing for any particular patient can be varied and will depend upon a variety of factors including the activity of the specific compound employed, the metabolic stability and length of action of that compound, the species, age, body weight, general health, sex and diet of the patient, the mode and time of administration, rate of excretion, drug combination, and severity of the particular condition. Preferred patients for treatment include animals, most preferably mammalian species such as humans, and domestic animals such as dogs, cats, and the like, patient to cancer.

As used herein, the terms “induction” and “induction phase” are used interchangeably and refer to the first phase of treatment in the clinical trial. For example, during induction, subjects may receive intravenous doses of an anti-PD1 antibody in combination with an anti-CS1 antibody.

As used herein, the terms “maintenance” and “maintenance phase” are used interchangeably and refer to the second phase of treatment in the clinical trial. For example, during maintenance, subjects may receive an anti-PD1 antibody in combination with an anti-CS1 antibody. In certain embodiments, treatment is continued as long as clinical benefit is observed or until unmanageable toxicity or disease progression occurs.

As used herein, the terms “fixed dose”, “flat dose” and “flat-fixed dose” are used interchangeably and refer to a dose that is administered to a patient without regard for the weight or body surface area (BSA) of the patient. The fixed or flat dose is therefore not provided as a mg/kg dose, but rather as an absolute amount of the agent (e.g., the anti-PD1 antibody and/or anti-CS1 antibody).

As used herein, a “body surface area (BSA)-based dose” refers to a dose (e.g., of the anti-PD1 antibody and/or anti-CS1 antibody) that is adjusted to the body-surface area (BSA) of the individual patient. A BSA-based dose may be provided as mg/kg body weight. Various calculations have been published to arrive at the BSA without direct measurement, the most widely used of which is the Du Bois formula (see Du Bois, D. et al., Archives of Internal Medicine, 17(6):863-871 (Jun. 1916); and Verbraecken, J. et al., Metabolism—Clinical and Experimental, 55(4):515-514 (Apr. 2006)). Other exemplary BSA formulas include the Mosteller formula (Mosteller, R. D., N Engl. J. Med., 317:1098 (1987)), the Haycock formula (Haycock, G. B. et al., J. Pediatr., 93:62-66 (1978)), the Gehan and George formula (Gehan, E. A. et al., Cancer Chemother. Rep., 54:225-235 (1970)), the Boyd formula (Current, J. D., The Internet Journal of Anesthesiology, 2(2) (1998); and Boyd, E., University of Minnesota, The Institute of Child Welfare, Monograph Series, No. 10., Oxford University Press, London (1935)), the Fujimoto formula (Fujimoto, S. et al., Nippon Eiseigaku Zasshi, 5:443-450 (1968)), the Takahira formula (Fujimoto, S. et al., Nippon Eiseigaku Zasshi, 5:443-450 (1968)), and the Schlich formula (Schlich, E. et al., Ernährungs Umschau, 57:178-183 (2010)).

The terms “combination” and “combinations” as used herein refer to either the concurrent administration of an anti-CS1 agent and an anti-PD1 agent; or to the sequential administration of an anti-CS1 agent with an anti-PD1 agent; or to the sequential administration of an anti-PD1 with an anti-CS1 agent; or to a more complex, combination, which may include for example, the combination of either an anti-CS1 agent and/or an anti-PD1 agent with another agent, such as an immunotherapeutic agent or co-stimulatory pathway modulator, preferably an agonist (i.e., immunostimulant), PROVENGE®, a tubulin stabilizing agent (e.g., paclitaxel, epothilone, taxane, etc.), Bevacizumab, IXEMPRA®, Dacarbazine, PARAPLATIN®, Docetaxel, one or more peptide vaccines, MDX-1379 Melanoma Peptide Vaccine, one or more gp100 peptide vaccine, fowlpox-PSA-TRICOM™ vaccine, vaccinia-PSA-TRICOM™ vaccine, MART-1 antigen, sargramostim, ticilimumab, Combination Androgen Ablative Therapy; the combination with a co-stimulatory pathway modulator; the combination with a tubulin stabilizing agent (e.g., paclitaxel, epothilone, taxane, etc.); the combination with IXEMPRA®, the combination with Dacarbazine, the combination with PARAPLATIN®, the combination with Docetaxel, the combination with one or more peptide vaccines, the combination with MDX-1379 Melanoma Peptide Vaccine, the combination with one or more gp100 peptide vaccine, the combination with fowlpox-PSA-TRICOM™ vaccine, the combination with vaccinia-PSA-TRICOM™ vaccine, the combination with MART-1 antigen, the combination with sargramostim, the combination with ticilimumab, and/or the combination with Combination Androgen Ablative Therapy. The combinations of the present invention may also be used in conjunction with other well-known therapies that are selected for their particular usefulness against the condition that is being treated. Such combinations may provide therapeutic options to those patients who present with more aggressive indications.

In another embodiment of the present invention, the combination between an anti-PD1 agent and anti-CS1 agent, may comprise at least one other agent, wherein said agent is selected from the following: a proteosome inhibitor (VELCADE®, KYPROLIS®, Ixazomib, etc.), an HDAC inhibitor (e.g., ISTODAX®, ZOLINZA®, Panobinostat, etc.), a CD anti-38 agent (e.g., Daratumumab), an anti-CD138 agent (e.g., Indatuximab), agatolimod, belatacept, blinatumomab, CD40 ligand, anti-B7-1 antibody, anti-B7-2 antibody, anti-B7-H4 antibody, AG4263, eritoran, anti-PD1 monoclonal antibodies, anti-OX40 antibody, ISF-154, and SGN-70.

In another embodiment of the present invention, the combination between an anti-PD1 agent and anti-CS1 agent, may comprise at least one other agent, wherein said agent is an IMiD, including but not limited to THALOMID® (thalidomide), REVLIMID® (lenalidomide), POMALYST® (pomalidomide), CC-120, CC-220, and CC-486 (Azacitidine). In specific embodiments, the present invention encompasses the following combinations: an anti-PD1 agent+an anti-CS1 agent+thalidomide; an anti-PD1 agent+an anti-CS1 agent+thalidomide+low-dose dexamethasone; an anti-PD1 agent+an anti-CS1 agent+thalidomide+high-dose dexamethasone; an anti-PD1 agent+an anti-CS1 agent+thalidomide+dexamethasone tablets; an anti-PD1 agent+an anti-CS1 agent+thalidomide+dexamethasone IV; an anti-PD1 agent+an anti-CS1 agent+lenalidomide; an anti-PD1 agent+an anti-CS1 agent+lenalidomide+low-dose dexamethasone; an anti-PD1 agent+an anti-CS1 agent+lenalidomide+high-dose dexamethasone; an anti-PD1 agent+an anti-CS1 agent+lenalidomide+dexamethasone tablets; an anti-PD1 agent+an anti-CS1 agent+lenalidomide+dexamethasone IV; an anti-PD1 agent+an anti-CS1 agent+pomalidomide; an anti-PD1 agent+an anti-CS1 agent+pomalidomide+low-dose dexamethasone; an anti-PD1 agent+an anti-CS1 agent+pomalidomide+high-dose dexamethasone; an anti-PD1 agent+an anti-CS1 agent+pomalidomide+dexamethasone tablets; an anti-PD1 agent+an anti-CS1 agent+pomalidomide+dexamethasone IV; wherein said anti-PD1 agent is an anti-PD1 agent disclosed herein, including nivolumab or pembrolizumab.

In another embodiment of the present invention, the combination between an anti-PD1 agent and an anti-CS1 agent, may comprise at least one other agent, wherein said at least one other agent is dexamethasone.

In another embodiment of the present invention, the combination between an anti-PD1 agent and an anti-CS1 agent, may comprise at least one other agent, wherein said at least one other agent is ipilimumab or tremelimumab.

In another embodiment of the present invention, the combination between an anti-PD1 agent and an anti-CS1 agent, may comprise at least one other agent, wherein said at least one other agent is ipilimumab or tremelimumab, and dexamethasone.

In another embodiment of the present invention, the combination between an anti-PD1 agent and an anti-CS1 agent, may comprise at least one other agent, wherein said at least one other agent is a chemotherapeutic agent.

A variety of chemotherapeutics are known in the art, some of which are described herein. One type of chemotherapeutic is referred to as a metal coordination complex. It is believed this type of chemotherapeutic forms predominantly inter-strand DNA cross links in the nuclei of cells, thereby preventing cellular replication. As a result, tumor growth is initially repressed, and then reversed. Another type of chemotherapeutic is referred to as an alkylating agent. These compounds function by inserting foreign compositions or molecules into the DNA of dividing cancer cells. As a result of these foreign moieties, the normal functions of cancer cells are disrupted and proliferation is prevented. Another type of chemotherapeutic is an antineoplastic agent. This type of agent prevents, kills, or blocks the growth and spread of cancer cells. Still other types of anticancer agents include nonsteroidal aromatase inhibitors, bifunctional alkylating agents, etc.

In another embodiment of the present invention, the chemotherapeutic agent may comprise microtubule-stabilizing agents, such as ixabepilone (IXEMPRA®) and paclitaxel (TAXOL®), which commonly are used for the treatment of many types of cancer and represent an attractive class of agents to combine with CTLA-4 blockade.

The phrase “microtubulin modulating agent” is meant to refer to agents that either stabilize microtubulin or destabilize microtubulin synthesis and/or polymerization.

One microtubulin modulating agent is paclitaxel (marketed as TAXOL®), which is known to cause mitotic abnormalities and arrest, and promotes microtubule assembly into calcium-stable aggregated structures resulting in inhibition of cell replication.

Epothilones mimic the biological effects of TAXOL®, (Bollag et al., Cancer Res., 55:2325-2333 (1995), and in competition studies act as competitive inhibitors of TAXOL® binding to microtubules. However, epothilones enjoy a significant advantage over TAXOL® in that epothilones exhibit a much lower drop in potency compared to TAXOL® against a multiple drug-resistant cell line (Bollag et al. (1995)). Furthermore, epothilones are considerably less efficiently exported from the cells by P-glycoprotein than is TAXOL® (Gerth (1996)). Additional examples of epothilones are provided in co-owned, PCT Application No. PCT/US2009/030291, filed Jan. 7, 2009, which is hereby incorporated by reference herein in its entirety for all purposes.

Ixabepilone is a semi-synthetic lactam analogue of patupilone that binds to tubulin and promotes tubulin polymerization and microtubule stabilization, thereby arresting cells in the G2/M phase of the cell cycle and inducing tumor cell apoptosis.

Additional examples of microtubule modulating agents useful in combination with immunotherapy include, but are not limited to, allocolchicine (NSC 406042), Halichondrin B (NSC 609395), colchicine (NSC 757), colchicine derivatives (e.g., NSC 33410), dolastatin 10 (NSC 376128), maytansine (NSC 153858), rhizoxin (NSC 332598), paclitaxel (TAXOL®, NSC 125973), TAXOL® derivatives (e.g., derivatives (e.g., NSC 608832), thiocolchicine NSC 361792), trityl cysteine (NSC 83265), vinblastine sulfate (NSC 49842), vincristine sulfate (NSC 67574), natural and synthetic epothilones including but not limited to epothilone A, epothilone B, epothilone C, epothilone D, desoxyepothilone A, desoxyepothilone B, [1S-[1R*,3R*(E),7R*,10S*,11R*,12R*,16S*]]-7-11-dihydroxy-8,8,10,12,16-pentamethyl-3-[1-methyl-2-(2-methyl-4-thiazolyl)ethenyl]-4-aza-17 oxabicyclo [14.1.0]heptadecane-5,9-dione (disclosed in U.S. Pat. No. 6,262,094, issued Jul. 17, 2001), [1S-[1R*,3R*(E),7R*,10S*,11R*,12R*,16S*]]-3-[2-[2-(aminomethyl)-4-thiazolyl]-1-methylethenyl]-7,11-dihydroxy-8,8,10,12,16-pentamethyl-4-17-dioxabicyclo [14.1.0]-heptadecane-5,9-dione (disclosed in U.S. patent application Ser. No. 09/506,481 filed on Feb. 17, 2000, and Examples 7 and 8 herein), and derivatives thereof; and other microtubule-disruptor agents. Additional antineoplastic agents include, discodermolide (see Service, Science, 274:2009 (1996)) estramustine, nocodazole, MAP4, and the like. Examples of such agents are also described in the scientific and patent literature, see, e.g., Bulinski, J. Cell Sci., 110:3055-3064 (1997); Panda, Proc. Natl. Acad. Sci. USA, 94:10560-10564 (1997); Muhlradt, Cancer Res., 57:3344-3346 (1997); Nicolaou, Nature, 387:268-272 (1997); Vasquez, Mol. Biol. Cell., 8:973-985 (1997); and Panda, J. Biol. Chem., 271:29807-29812 (1996).

The following sets forth preferred therapeutic combinations and exemplary dosages for use in the methods of the present invention.

| | Dosage | |
|---|---|---|
| Therapeutic Combination(s) | mg/kg (per dose) | |
| Anti-CS1 antibody + | 1-10 | mg/kg |
| Anti-PD1 Antibody | 0.1-1 | mg/kg |
| Anti-CS1 antibody + | 10 | mg/kg |
| Anti-PD1 Antibody | 1 | mg/kg |
| Anti-CS1 antibody + | 10 | mg/kg |
| Anti-PD1 Antibody | 3 | mg/kg |
| Anti-CS1 antibody + | 10 | mg/kg |
| Anti-PD1 Antibody | 0.3 | mg/kg |
| Anti-CS1 antibody + | 10 | mg/kg |
| Anti-PD1 Antibody | 0.1 | mg/kg |
| Anti-CS1 antibody + | 1 | mg/kg |
| Anti-PD1 Antibody | 1 | mg/kg |
| Anti-CS1 antibody + | 1 | mg/kg |
| Anti-PD1 Antibody | 3 | mg/kg |
| Anti-CS1 antibody + | 1 | mg/kg |
| Anti-PD1 Antibody | 0.3 | mg/kg |
| Anti-CS1 antibody + | 1 | mg/kg |
| Anti-PD1 Antibody | 0.1 | mg/kg |
| Anti-CS1 antibody + | 1 | mg/kg |
| Anti-PD1 Antibody | 0.03 | mg/kg |
| Anti-CS1 antibody + | 10 | mg/kg |
| Anti-PD1 Antibody | 0.03 | mg/kg |
| Anti-CS1 antibody + | 1 | mg/kg |
| Anti-PD1 Antibody | 3 | mg |
| Anti-CS1 antibody + | 10 | mg/kg |
| Anti-PD1 Antibody | 3 | mg |
| Anti-CS1 antibody + | 1 | mg/kg |
| Anti-PD1 Antibody | 8 | mg |
| Anti-CS1 antibody + | 10 | mg/kg |
| Anti-PD1 Antibody | 8 | mg |

While this table provides exemplary dosage ranges of the anti-CS1 and anti-PD1 antibodies, when formulating the pharmaceutical compositions of the invention the clinician may utilize preferred dosages as warranted by the condition of the patient being treated. For example, Elotuzumab may preferably be administered at about 10 mg/kg every 3 weeks. Nivolumab may preferably be administered at about 0.03, 0.1, 1, 3, 0.1-10 mg/kg, or 3 or 8 kg, every three weeks.

The anti-CS1 antibody may preferably be administered at about 0.1-20 mg/kg, or the maximum tolerated dose. In an embodiment of the invention, a dosage of anti-CS1 antibody is administered about every three weeks. Alternatively, the anti-CS1 antibody may be administered by an escalating dosage regimen including administering a first dosage of anti-CS1 antibody at about 1 mg/kg, a second dosage of anti-CS1 antibody at about 3 mg/kg, and a third dosage of anti-CS1 antibody at about 10 mg/kg.

In another specific embodiment, the escalating dosage regimen includes administering a first dosage of anti-CS1 antibody at about 3 mg/kg and a second dosage of anti-CS1 antibody at about 10 mg/kg.

The anti-PD1 antibody may preferably be administered at about 0.03, 1 mg/kg, 3 mg/kg, up to 20 mg/kg, or the maximum tolerated dose. In an embodiment of the invention, a dosage of anti-PD1 antibody is administered about every three weeks. Alternatively, the anti-PD1 antibody may be administered by an escalating dosage regimen including administering a first dosage of anti-PD1 antibody at about 0.1 mg/kg, a second dosage of anti-PD1 antibody at about 0.3 mg/kg, and a third dosage of anti-PD1 antibody at about 1 mg/kg. Alternatively, the anti-PD1 antibody may be administered by an escalating dosage regimen including administering a first dosage of anti-PD1 antibody at about 0.3 mg/kg, a second dosage of anti-PD1 antibody at about 1 mg/kg, and a third dosage of anti-PD1 antibody at about 3 mg/kg.

In another specific embodiment, the escalating dosage regimen includes administering a first dosage of anti-PD1 antibody at about 1 mg/kg and a second dosage of anti-PD1 antibody at about 3 mg/kg.

In another specific embodiment, the escalating dosage regimen includes administering a first dosage of anti-PD1 antibody at about 3 mg and a second dosage of anti-PD1 antibody at about 8 mg.

Further, the present invention provides an escalating dosage regimen, which includes administering an increasing dosage of anti-CS1 antibody about every six weeks.

In one embodiment, the anti-CS1 antibody is administered on (1) day 1, week 1, (2) day 1, week 2, (3) day 1, week 3, (4) day 1, week 4, (5) day 1, week 5, (6) day 1, week 6, (7) day 1, week 7, and (8) day 1, week 8, of the induction phase. In another embodiment, the anti-PD1 antibody is administered on (1) day 1, week 1, (2) day 1, week 4, and (3) day 1, week 7 of the induction phase. In another embodiment, the anti-CS1 antibody is administered on (1) day 1, week 10 and (2) day 1, week 15 of the maintenance phase. In another embodiment, the anti-PD1 antibody is administered on (1) day 1, week 10 of the maintenance phase. In another embodiment, the maintenance phase is repeated for at least 1, 2, 3, 4, 5, 6, 7, 8, 9, 10, 11, 12, 13, 14, 15, 16, 17, 18, 19, or 20 or more cycles.

The actual dosage employed may be varied depending upon the requirements of the patient and the severity of the condition being treated. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired. Intermittent therapy (e.g., one week out of three weeks or three out of four weeks) may also be used.

In certain specific embodiments, the anti-CS1 antibody and anti-PD-1 antibody are administered according to one of the following dosing regimens:

(a) 10 mg of the anti-CS1 antibody weekly for 4 weeks and 3 mg/kg of the anti-PD-1 antibody every 2 weeks;

(b) 10 mg of the anti-CS1 antibody weekly for 4 weeks and 1 mg/kg of the anti-PD-1 antibody every 2 weeks;

(c) 10 mg of the anti-CS1 antibody every 2 weeks and 3 mg/kg of the anti-PD-1 antibody every 3 weeks; and

(d) 10 mg of the anti-CS1 antibody every 3 weeks and 3 mg/kg of the anti-PD-1 antibody every 2 weeks.

The anti-PD1 antibody may be administered once every two weeks after the initial treatment cycle until disease progression or unacceptable toxicity.

In other embodiments, the anti-CS1 antibody and anti-PD-1 antibody may be combined with an anti-CTLA4 antibody (e.g., ipilimumab or tremelimumab), and administered according to one of the following dosing regimens:

(a) 10 mg of the anti-CS1 antibody weekly for 4 weeks and 1 mg/kg of the anti-CTLA4 antibody and 3 mg/kg of the anti-PD-1 antibody every 3 weeks;

(b) 10 mg of the anti-CS1 antibody every 2 weeks for 4 doses and 1 mg/kg of the anti-CTLA4 antibody and 3 mg/kg of the anti-PD-1 antibody every 3 weeks;

(c) 10 mg of the anti-CS1 antibody weekly for 4 weeks and 1 mg/kg of the anti-CTLA4 antibody and 3 mg/kg of the anti-PD-1 antibody every 2 weeks; and

(d) 10 mg of the anti-CS1 antibody weekly for 3 weeks and 1 mg/kg of the anti-CTLA4 antibody and 3 mg/kg of the anti-PD-1 antibody every 2 weeks The anti-PD1 antibody may be administered once every two weeks after the initial treatment cycle until disease progression or unacceptable toxicity.

For combinations encompassing the addition of an IMiD, it would be within the skill of the prescribing physician to provide a recommended dose for treatment. Suggested doses for thalidomide include: 50 mg, 100 mg, or 200 mg, and when administered as part of a 1 month cycle, administering thalidomide on days 1 to 14. Suggested doses for lenalidomide include 25 mg once daily, and when administered as part of a 1 month cycle, administering lenalidomide on days 1 to 21. Suggested doses for pomalidomide include 1 mg, 2 mg, 3 mg, or 4 mg once daily, and when administered as part of a 1 month cycle, administering pomalidomide on days 1 to 21.

For combinations encompassing the addition of dexamethasone, it would be within the skill of the prescribing physician to provide a recommended dose for treatment. Suggested doses for low-dose dexamethasone include: 28 mg once daily, and when administered as part of a 1 month cycle, administering low-dose dexamethasone on days 1, 8, 15, and 22 (for cycles 1 and 2); on days 1 and 15 (cycles 3 to 18); and day 1 (cycle 19 and beyond). Suggested doses for high-dose dexamethasone include: 40 mg once daily, and when administered as part of a 1 month cycle, administering low-dose dexamethasone on days 8 and 22 (for cycles 3 to 18); and on days 8, 15, and 22 (cycles 19 and beyond). Suggested doses for IV dexamethasone include: 8 mg IV once daily, and when administered as part of a 1 month cycle, administering IV dexamethasone on days 1, 8, 15, and 22 (for cycles 1 and 2); on days 1 and 15 (cycles 3 to 18) and on day 1 (cycles 19 and beyond).

In practicing the many aspects of the invention herein, biological samples can be selected preferably from blood, blood cells (red blood cells or white blood cells). Cells from a sample can be used, or a lysate of a cell sample can be used. In certain embodiments, the biological sample comprises blood cells.

Pharmaceutical compositions for use in the present invention can include compositions comprising one or a combination of co-stimulatory pathway modulators in an effective amount to achieve the intended purpose. A therapeutically effective dose refers to that amount of active ingredient which ameliorates the symptoms or condition. Therapeutic efficacy and toxicity in humans can be predicted by standard pharmaceutical procedures in cell cultures or experimental animals, for example the ED50 (the dose therapeutically effective in 50% of the population) and LD50 (the dose lethal to 50% of the population).

A “therapeutically effective amount” of either an anti-PD1 agent or an anti-CS1 agent may range anywhere from 1 to 14 fold or more higher than the typical dose depending upon the patients indication and severity of disease. Accordingly, therapeutically relevant doses of an anti-PD1 agent or an anti-CS1 agent for any disorder disclosed herein can be, for example, about 1, 2, 3, 4, 5, 6, 7, 8, 9, 10, 11, 12, 13, 14, 15, 16, 17, 18, 19, 20, 25, 30, 35, 40, 50, 60, 70, 80, 90, 100, 125, 150, 175, 200, 225, 250, or 300 fold higher than the prescribed or standard dose. Alternatively, therapeutically relevant doses of an anti-PD1 agent or an anti-CS1 agent can be, for example, about 1.0×, about 0.9×, 0.8×, 0.7×, 0.6×, 0.5×, 0.4×, 0.3×, 0.2×, 0.1×, 0.09×, 0.08×, 0.07×, 0.06×, 0.05×, 0.04×, 0.03×, 0.02×, or 0.01×.

Disorders for which the sequential dosing regimen may be useful in treating includes one or more of the following disorders: melanoma, prostate cancer, and lung cancer, for example, also include leukemias, including, for example, chronic myeloid leukemia (CML), acute lymphoblastic leukemia, and Philadelphia chromosome positive acute lymphoblastic leukemia (Ph+ ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia, chronic lymphocytic leukemia, mastocytosis and any symptom associated with mastocytosis. In addition, disorders include urticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, and mast cell leukemia. Various additional cancers are also included within the scope of protein tyrosine kinase-associated disorders including, for example, the following: carcinoma, including that of the bladder, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burkitt's lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma. In certain preferred embodiments, the disorder is leukemia, breast cancer, prostate cancer, lung cancer, colon cancer, melanoma, or solid tumors. In certain preferred embodiments, the leukemia is chronic myeloid leukemia (CML), Ph+ ALL, AML, imatinib-resistant CML, imatinib-intolerant CML, accelerated CML, lymphoid blast phase CML.

The terms “cancer”, “cancerous”, or “malignant” refer to or describe the physiological condition in mammals, or other organisms, that is typically characterized by unregulated cell growth. Examples of cancer include, for example, solid tumors, melanoma, leukemia, lymphoma, blastoma, carcinoma and sarcoma. More particular examples of such cancers include chronic myeloid leukemia, acute lymphoblastic leukemia, Philadelphia chromosome positive acute lymphoblastic leukemia (Ph+ ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia (AML), and chronic lymphocytic leukemia (CML).

A “solid tumor” includes, for example, sarcoma, melanoma, colon carcinoma, breast carcinoma, prostate carcinoma, or other solid tumor cancer.

“Leukemia” refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease—acute or chronic; (2) the type of cell involved; myeloid (myelogenous), lymphoid (lymphogenous), or monocytic; and (3) the increase or non-increase in the number of abnormal cells in the blood—leukemic or aleukemic (subleukemic). Leukemia includes, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia. In certain aspects, the present invention provides treatment for chronic myeloid leukemia, acute lymphoblastic leukemia, and/or Philadelphia chromosome positive acute lymphoblastic leukemia (Ph+ ALL).

Provided herein are methods for treating cancer (e.g., hematological cancers, including Multiple Myeloma) in a patient comprising administering to the patient an anti-CS1 antibody and an anti-PD1 antibody. Preferably, the combination therapy exhibits therapeutic synergy.

“Therapeutic synergy” refers to a phenomenon where treatment of patients with a combination of therapeutic agents manifests a therapeutically superior outcome to the outcome achieved by each individual constituent of the combination used at its optimum dose (Corbett, T. H. et al., Cancer Treatment Reports, 66:1187 (1982)). For example, a therapeutically superior outcome is one in which the patients either a) exhibit fewer incidences of adverse events while receiving a therapeutic benefit that is equal to or greater than that where individual constituents of the combination are each administered as monotherapy at the same dose as in the combination, or b) do not exhibit dose-limiting toxicities while receiving a therapeutic benefit that is greater than that of treatment with each individual constituent of the combination when each constituent is administered in at the same doses in the combination(s) as is administered as individual components. Accordingly, in one embodiment, administration of the anti-PD1 antibody and anti-CS1 antibodies has a synergistic effect on treatment compared to administration of either antibody alone.

Alternatively, the combination therapy of an anti-CS1 antibody and an anti-PD1 antibody may have an additive or superadditive effect on suppressing cancer (e.g., Multiple Myeloma), as compared to monotherapy with either antibody alone. By “additive” is meant a result that is greater in extent than the best separate result achieved by monotherapy with each individual component, while “superadditive” is used to indicate a result that exceeds in extent the sum of such separate results. In one embodiment, the additive effect is measured as, e.g., reduction in paraproteins, reduction of plasmacytosis, reduction of bone lesions over time, increase in overall response rate, or increase in median or overall survival.

Multiple Myeloma disease response or progression, in particular, is typically measured according to the size of reduction (or rise) in paraproteins. However, the degree of plasmacytosis in the bone marrow (increase in percentage of plasma cells in the bone marrow), progression of bone lesions, and the existence of soft tissue plasmacytomas (a malignant plasma cell tumor growing within soft tissue) are also considered (Smith, D. et al., BMJ, 346:f3863 (Jun. 26, 2013)). Responses to therapy may include:

Complete Response
No detectable paraprotein and disappearance of any soft tissue
plasmacytomas and <5% plasma cells in bone marrow.
Very Good Partial Response
Greater than 90% reduction in paraproteins or paraproteins detectable but
too low to measure.
Partial Response
Greater than 50% reduction in paraproteins.
No Change or Stable Disease
Not meeting criteria for disease response or progression.
Progressive Disease
At least a 25% increase in paraproteins (increase of at least 5 g/L),
development of new bone lesions or plasmacytomas, or hypercalcaemia.
(corrected serum calcium >2.65 mmol/L)

Patients treated according to the methods disclosed herein preferably experience improvement in at least one sign of Multiple Myeloma. In one embodiment, the patient treated exhibits a complete response (CR), a very good partial response (VGPR), a partial response (PR), or stable disease (SD).

In one embodiment, improvement is measured by a reduction in paraprotein and/or decrease or disappearance of soft tissue plasmacytomas. In another embodiment, lesions can be measured by radiography. In another embodiment, cytology or histology can be used to evaluate responsiveness to a therapy.

In other embodiments, administration of effective amounts of the anti-PD1 antibody and anti-CS1 antibody according to any of the methods provided herein produces at least one therapeutic effect selected from the group consisting of reduction in paraprotein, decrease or disappearance of soft tissue plasmacytomas, CR, VGPR, PR, or SD. In still other embodiments, the methods of treatment produce a comparable clinical benefit rate (CBR =CR+PR+SD >6 months) better than that achieved by an anti-PD1 antibody or anti-CS1 antibody alone. In other embodiments, the improvement of clinical benefit rate is about 20% 20%, 30%, 40%, 50%, 60%, 70%, 80% or more compared to an anti-PD1 antibody or anti-CS1 antibody alone.

### Antibodies

The term “antibody” describes polypeptides comprising at least one antibody derived antigen binding site (e.g., VH/VL region or Fv, or CDR). Antibodies include known forms of antibodies. For example, the antibody can be a human antibody, a humanized antibody, a bispecific antibody, or a chimeric antibody. The antibody also can be a Fab, Fab′2, ScFv, SMIP, AFFIBODY®, nanobody, or a domain antibody. The antibody also can be of any of the following isotypes: IgG1, IgG2, IgG3, IgG4, IgM, IgA1, IgA2, IgAsec, IgD, and IgE. The antibody may be a naturally occurring antibody or may be an antibody that has been altered (e.g., by mutation, deletion, substitution, conjugation to a non-antibody moiety). For example, an antibody may include one or more variant amino acids (compared to a naturally occurring antibody) which changes a property (e.g., a functional property) of the antibody. For example, numerous such alterations are known in the art which affect, e.g., half-life, effector function, and/or immune responses to the antibody in a patient. The term antibody also includes artificial polypeptide constructs which comprise at least one antibody-derived antigen binding site.

Antibodies also include known forms of antibodies. For example, the antibody can be a human antibody, a humanized antibody, a bispecific antibody, or a chimeric antibody. The antibody also can be a Fab, Fab′2, ScFv, SMIP, AFFIBODY®, nanobody, or a domain antibody. The antibody also can be of any of the following isotypes: IgG1, IgG2, IgG3, IgG4, IgM, IgA1, IgA2, IgAsec, IgD, and IgE. The antibody may be a naturally occurring antibody or may be an antibody that has been altered (e.g., by mutation, deletion, substitution, conjugation to a non-antibody moiety). For example, an antibody may include one or more variant amino acids (compared to a naturally occurring antibody) which changes a property (e.g., a functional property) of the antibody. For example, numerous such alterations are known in the art which affect, e.g., half-life, effector function, and/or immune responses to the antibody in a patient. The term antibody also includes artificial polypeptide constructs which comprise at least one antibody-derived antigen binding site.

The concurrent dosing regimen of the present invention may include the use of antibodies as one component of the combination. For example, antibodies that specifically bind to CS-1 polypeptides, preferably Elotuzumab, and/or PD1, preferably Nivolumab.

Alternatively, the sequential dosing regimen of the present invention may include the use of antibodies as one component of the combination. For example, antibodies that specifically bind to CS-1 polypeptides, preferably Elotuzumab, and/or PD1, preferably Nivolumab.

The term “antibody” is also used in the broadest sense and specifically covers monoclonal antibodies, polyclonal antibodies, antibody compositions with polyepitopic specificity, bispecific antibodies, diabodies, chimeric, single-chain, and humanized antibodies, as well as antibody fragments (e.g., Fab, F(ab′)₂, and Fv), so long as they exhibit the desired biological activity. Antibodies can be labeled for use in biological assays (e.g., radioisotope labels, fluorescent labels) to aid in detection of the antibody.

Antibodies can be prepared using, for example, intact polypeptides or fragments containing small peptides of interest, which can be prepared recombinantly for use as the immunizing antigen. The polypeptide or oligopeptide used to immunize an animal can be derived from the translation of RNA or synthesized chemically, and can be conjugated to a carrier protein, if desired. Commonly used carriers that are chemically coupled to peptides include, for example, bovine serum albumin (BSA), keyhole limpet hemocyanin (KLH), and thyroglobulin. The coupled peptide is then used to immunize the animal (e.g., a mouse, a rat, or a rabbit).

The term “antigenic determinant” refers to that portion of a molecule that makes contact with a particular antibody (i.e., an epitope). When a protein or fragment of a protein is used to immunize a host animal, numerous regions of the protein can induce the production of antibodies that bind specifically to a given region or three-dimensional structure on the protein; each of these regions or structures is referred to as an antigenic determinant. An antigenic determinant can compete with the intact antigen (i.e., the immunogen used to elicit the immune response) for binding to an antibody.

The phrase “specifically binds to” refers to a binding reaction that is determinative of the presence of a target in the presence of a heterogeneous population of other biologics. Thus, under designated assay conditions, the specified binding region binds preferentially to a particular target and does not bind in a significant amount to other components present in a test sample. Specific binding to a target under such conditions can require a binding moiety that is selected for its specificity for a particular target. A variety of assay formats can be used to select binding regions that are specifically reactive with a particular analyte. Typically a specific or selective reaction will be at least twice background signal or noise and more typically more than 10 times background.

### Anti-CS1 Antibodies

Anti-human-CS1 antibodies (or VH and/or VL domains derived therefrom) suitable for use in the invention can be generated using methods well known in the art. Alternatively, art recognized anti-CS1 antibodies can be used. For example, the monoclonal antibody mAb 162 described in Bouchon et al., J. Immunol., 167:5517-5521 (2001) can be used, the teachings of which are hereby incorporated by reference herein in their entirety, and in particular, those portions directly related to this antibody. Another known CS1 antibody includes the anti-CS1 antibody described in Matthew et al. (U.S. Pat. No. 7,041,499), the teachings of which are hereby incorporated by reference herein in their entirety, and in particular, those portions directly related to this antibody. Other known CS1 antibodies include the anti-CS1 antibody, Luc 63 and other antibodies that share the same epitope, including Luc 4, Luc 12, Luc 23, Luc 29, Luc 32 and Luc 37, the anti-CS1 antibody Luc 90 and other antibodies that share the same epitope, including Luc 34, Luc 69 and Luc X, and the anti-CS1 antibodies Luc2, Luc3, Luc15, Luc22, Luc35, Luc38, Luc39, Luc56, Luc60, LucX.1, LucX.2, and PDL-241, described in Williams et al. (U.S. Pat. No. 7,709,610), the teachings of which are hereby incorporated by reference herein in their entirety, and in particular, those portions directly related to these antibodies. Antibodies that compete with any of these art-recognized antibodies for binding to CS1 also can be used.

An exemplary anti-CS1 antibody is Elotuzumab (also referred to as BMS-901608 and HuLuc63) comprising heavy and light chains having the sequences shown in

SEQ ID NOs:17 and 18, respectively, or antigen binding fragments and variants thereof. Elotuzumab is a humanized IgG anti-CS-1 monoclonal antibody described in PCT Publication Nos. WO 2004/100898, WO 2005/10238, WO 2008/019376, WO 2008/019378, WO 2008/019379, WO 2010/051391, WO 2011/053321, and WO 2011/053322, the teachings of which are hereby incorporated by reference. Elotuzumab is known to mediate ADCC through NK cells (van Rhee, F. et al., Mol. Cancer Ther., 8(9):2616-2624 (2009)).

In other embodiments, the antibody comprises the heavy and light chain CDRs or variable regions of Elotuzumab. Accordingly, in one embodiment, the antibody comprises the CDR1, CDR2, and CDR3 domains of the VH of Elotuzumab having the sequence set forth in SEQ ID NO:2, and the CDR1, CDR2 and CDR3 domains of the VL of Elotuzumab having the sequences set forth in SEQ ID NO:1. In another embodiment, the antibody comprises heavy chain CDR1 having amino acids 31-35 of SEQ ID NO:2: a heavy chain CDR2 having amino acids 50-66 of SEQ ID NO:2; and a heavy chain CDR3 having amino acids 99-108 of SEQ ID NO:2; in addition to a light chain CDR1 having amino acids 24-34 of SEQ ID NO:1; a light chain CDR2 having amino acids 50-56 of SEQ ID NO:1; and a light chain CDR3 having amino acids 89-97 of SEQ ID NO:1. In another embodiment, the antibody comprises VH and/or VL regions having the amino acid sequences set forth in SEQ ID NO: 2 and/or SEQ ID NO: 1, respectively. In another embodiment, the antibody competes for binding with and/or binds to the same epitope on CS1 as the above-mentioned antibodies. In another embodiment, the antibody has at least about 90% variable region amino acid sequence identity with the above-mentioned antibodies (e.g., at least about 90%, 95% or 99% variable region identity with SEQ ID NO:2 or SEQ ID NO:1).

### Anti-PD1 Antibodies

HuMAbs that bind specifically to PD-1 with high affinity have been disclosed in U.S. Pat. No. 8,008,449. Other anti-PD-1 mAbs have been described in, for example, U.S. Pat. Nos. 6,808,710, 7,488,802, 8,168,757 and 8,354,509, and PCT Publication No. WO 2012/145493. Each of the anti-PD-1 HuMAbs disclosed in U.S. Pat. No. 8,008,449 has been demonstrated to exhibit one or more of the following characteristics: (a) binds to human PD-1 with a KD of 1×10⁻⁷ M or less, as determined by surface plasmon resonance using a BIACORE® biosensor system; (b) does not substantially bind to human CD28, CTLA-4 or ICOS; (c) increases T-cell proliferation in a Mixed Lymphocyte Reaction (MLR) assay; (d) increases interferon-γ production in an MLR assay; (e) increases IL-2 secretion in an MLR assay; (f) binds to human PD-1 and cynomolgus monkey PD-1; (g) inhibits the binding of PD-L1 and/or PD-L2 to PD-1; (h) stimulates antigen-specific memory responses; (i) stimulates Ab responses; and (j) inhibits tumor cell growth in vivo. Anti-PD-1 Abs usable in the present invention include mAbs that bind specifically to human PD-1 and exhibit at least one, preferably at least five, of the preceding characteristics.

A preferred anti-PD-1 Ab is nivolumab (also referred to as BMS-936558). Nivolumab is a fully human IgG4 anti-PD-1 monoclonal antibody disclosed as 5C4 in WO 2006/121168. Nivolumab is known to augment cellular immune responses against tumors (Brahmer, J. R. et al., J. Clin. Oncol., 28:3167-3175 (2010)). Another anti-PD-1 Ab usable in the present methods is pembrolizumab (Hamid et al., N. Engl. J. Med., 369(2):134-144 (2013)).

Anti-PD-1 Abs usable in the disclosed methods also include isolated Abs that bind specifically to human PD-1 and cross-compete for binding to human PD-1 with nivolumab (see, e.g., U.S. Pat. No. 8,008,449; WO 2013/173223). The ability of Abs to cross-compete for binding to an antigen indicates that these Abs bind to the same epitope region of the antigen and sterically hinder the binding of other cross-competing Abs to that particular epitope region. These cross-competing Abs are expected to have functional properties very similar those of nivolumab by virtue of their binding to the same epitope region of PD-1. Cross-competing Abs can be readily identified based on their ability to cross-compete with nivolumab in standard PD-1 binding assays such as BIACORE® analysis, ELISA assays or flow cytometry (see, e.g., WO 2013/173223).

For administration to human subjects, these anti-PD-1 Abs are preferably chimeric Abs, or more preferably humanized or human Abs. Such chimeric, humanized or human mAbs can be prepared and isolate 5 d by methods well known in the art. Anti-PD-1 Abs usable in the methods of the disclosed invention also include antigen-binding portions of the above Abs. It has been amply demonstrated that the antigen-binding function of an Ab can be performed by fragments of a full-length Ab. Examples of binding fragments encompassed within the term “antigen-binding portion” of an Ab include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; and (iv) a Fv fragment consisting of the VL and VH domains of a single arm of an Ab. Anti-PD-1 antibodies (or VH and/or VL domains derived therefrom) suitable for use in the invention can be generated using methods well known in the art.

An exemplary anti-PD-1 antibody is nivolumab comprising heavy and light chains comprising the sequences shown in SEQ ID NOs: 4 and 3, respectively, or antigen binding fragments and variants thereof.

In other embodiments, the antibody has heavy and light chain CDRs or variable regions of nivolumab. Accordingly, in one embodiment, the antibody comprises CDR1, CDR2, and CDR3 domains of the VH of nivolumab having the sequence set forth in SEQ ID NO: 4, and CDR1, CDR2 and CDR3 domains of the VL of nivolumab having the sequence set forth in SEQ ID NO: 3. In another embodiment, the antibody comprises a light chain CDR1 having amino acids 24-34 of SEQ ID NO:3, a light chain CDR2 having amino acids 50-56 of SEQ ID NO:3, and a light chain CDR3 having amino acids 89-97 of SEQ ID NO:3; and comprising a heavy chain CDR1 having amino acids 31-35 of SEQ ID NO:4, a heavy chain CDR2 having amino acids 50-66 of SEQ ID NO:4, and a heavy chain CDR3 having amino acids 99-102 of SEQ ID NO:4. In another embodiment, the antibody comprises VH and/or VL regions comprising the amino acid sequences set forth in SEQ ID NO: 4 and/or SEQ ID NO: 3, respectively. In another embodiment, the antibody competes for binding with and/or binds to the same epitope on PD-1 as the above-mentioned antibodies. In another embodiment, the antibody has at least about 90% variable region amino acid sequence identity with the above-mentioned antibodies (e.g., at least about 90%, 95% or 99% variable region identity with SEQ ID NO: 3 or SEQ ID NO: 4).

### Kits

For use in the diagnostic and therapeutic applications described or suggested above, kits are also provided by the invention. Such kits can, for example, comprise a carrier means being compartmentalized to receive in close confinement one or more container means such as vials, tubes, and the like, each of the container means comprising one of the separate elements to be used in the method. For example, one of the container means can comprise one or more vials containing a pharmaceutically acceptable amount of an anti-CS1 antibody, and/or an anti-PD1 antibody.

The kit of the invention will typically comprise the container described above and one or more other containers comprising materials desirable from a commercial and user standpoint, including buffers, diluents, filters, needles, syringes, and package inserts with instructions for use. A label can be present on the container to indicate that the composition is used for a specific therapy or non-therapeutic application, and can also indicate directions for either in vivo or in vitro use, such as those described above.

In addition, the kits can include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips, and the like), optical media (e.g., CD-ROM), and the like. Such media can include addresses to internet sites that provide such instructional materials.

The kit can also comprise, for example, a means for obtaining a biological sample from an individual. Means for obtaining biological samples from individuals are well known in the art, e.g., catheters, syringes, and the like, and are not discussed herein in detail.

Also provided herein are kits which include a pharmaceutical composition containing an anti-PD1 antibody, such as nivolumab, and an anti-CS1 antibody, such as Elotuzumab, and a pharmaceutically-acceptable carrier, in a therapeutically effective amount adapted for use in the preceding methods. The kits optionally also can include instructions, e.g., comprising administration schedules, to allow a practitioner (e.g., a physician, nurse, or patient) to administer the composition contained therein to administer the composition to a patient having cancer (e.g., a hematological cancer, such as Multiple Myeloma). The kit also can include a syringe.

Optionally, the kits include multiple packages of the single-dose pharmaceutical compositions each containing an effective amount of the anti-PD1 antibody or anti-CS1 antibody for a single administration in accordance with the methods provided above. Instruments or devices necessary for administering the pharmaceutical composition(s) also may be included in the kits. For instance, a kit may provide one or more pre-filled syringes containing an amount of the anti-PD1 antibody or anti-CS1 antibody.

In one embodiment, the present invention provides a kit for treating a cancer (e.g., a hematological cancer, such as Multiple Myeloma) in a human patient, the kit comprising:

(a) a dose of an anti-PD1 antibody comprising the CDR1, CDR2 and CDR3 domains in a heavy chain variable region comprising the sequence set forth in SEQ ID NO:3, and the CDR1, CDR2 and CDR3 domains in a light chain variable region comprising the sequence set forth in SEQ ID NO:3;

(b) a dose of an anti-CS1 antibody comprising the CDR1, CDR2 and CDR3 domains in a heavy chain variable region comprising the sequence set forth in SEQ ID NO:2, and the CDR1, CDR2 and CDR3 domains in a light chain variable region comprising the sequence set forth in SEQ ID NO:11; and

(c) instructions for using the anti-PD1 antibody and anti-CS1 antibody in the methods described herein.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

The following representative Examples contain important additional information, exemplification and guidance which can be adapted to the practice of this invention in its various embodiments and the equivalents thereof. These examples are intended to help illustrate the invention, and are not intended to, nor should they be construed to, limit its scope.

### REFERENCES

1) Li, B. et al., “Anti-programmed death-1 synergizes with granulocyte macrophage colony-stimulating factor-secreting tumor cell immunotherapy providing therapeutic benefit to mice with established tumors”, Clin. Cancer Res., 15:1623-1634 (Mar. 1, 2009).
2) Fransen, M. F. et al., “Controlled local delivery of CTLA-4 blocking antibody induces CD8 T cell dependent tumor eradication and decreases risk of toxic side effects”, Clin. Cancer Res. (2013).

### Materials and Methods

### Cell Lines

pFB-GFP or pFB-hSLAMF7-GFP plasmids were transfected into Phoenix cells using Lipo2000 (Invitrogen). A20 or EG7 cells were transduced with pFB-GFP or pFB-hSLAMF7-GFP virus with polybrene (Sigma) by two rounds of spin infection at 2500 rpm for 90 min at room temperature. Individual clones were selected and expanded. Prior to use in animal studies, A20-GFP, A20-hSLAMF7-GFP, EG7-GFP, and EG7-hSLAMF7-GFP cell lines were analyzed for mycoplasma and pathogens (RADIL testing).

### Mice

Mice used for all in vivo studies were eight- to ten-week old Balb/c or C57BL/6 mice obtained from either Charles River, Taconic or Jackson Labs. Studies were performed according to the standards of “Guide for the Care and Use of Laboratory Animals” using protocols approved by IACUC.

### Antibodies

Elotuzumab is a humanized anti-human SLAMF7 antibody, IgG1 (formerly HuLuc63). To make Elotuzumab with the mouse IgG2a isotype, the VH from plasmid #303 pMuLuc63 (obtained from AbbVie) was amplified and cloned into an expression vector containing the mouse IgG2a constant region to produce pICOFSCpur.mg2a (CS1.1). The VK from plasmid #303 pMuLuc63 was amplified and cloned into an expression vector containing the mouse kappa constant region to produce pICOFSCneo.mK (CS1.1). The two vectors were co-transfected into CHO-S cells and stable clones were selected. CHO-S clone CS1.1-mg2a #9G4 termed Elo-mIgG2a was scaled up for antibody production. Anti-mouse PD-1 antibody, 4H2, was generated by immunization of rats with mouse PD-1-immunoglobulin fusion protein (Li, B. et al., Clin. Cancer Res., 15:1623-1634 (2009)). Binding of the antibody to mouse PD-1 was shown by ELISA to PD-1-immunoglobulin fusion and by flow cytometry with transfected Chinese hamster ovary cells expressing mouse PD-1. The antibody was selected for its ability to inhibit the interaction between mouse PD-land its ligand PD-L1 or PD-L2. The variable (V) region sequences of this antibody were determined and VH and VK sequences were grafted onto the murine IgG1 constant region containing the D265A mutation to eliminate Fc receptor binding (PD-1-4H2-mg1-D265A). Chinese hamster ovary cell lines that express the chimeric antibody were selected and used for production of the antibody. Control antibodies include anti-mIgG2a (clone C1.18.4, Bioxcell) and anti-mIgG1, anti-diphtheria toxin antibody with a mouse IgG1 isotype (BMS).

### Cell Culturing Conditions

A20 cells were cultured in RPMI medium (Gibco) supplemented with 10% of Fetal Bovine Serum (FBS), 0.05 mM 2-mercaptoethanol; EG7 cells were cultured in RPMI medium supplemented with 2 mM L-glutamine, 10% FBS, 1.5 g/L sodium bicarbonate, 4.5 g/L glucose, 10 mM HEPES, 1 mM sodium pyruvate, 0.05 mM 2-mercaptoethanol, 0.4 mg/ml G418 (EG7). Cells were passaged three times a week and maintained at a concentration of 0.3×106 cells/ml

### Tumor Studies

A20 tumors were established via subcutaneous injection of 1×10⁷ A20-GFP or A20-hSLAMF7-GFP cells into hind flank of mice. After 10-17 days, tumor volumes were determined and mice were randomized into treatment groups when the average tumor volume reached 150-180 mm³. EG7 tumors were established via subcutaneous injection of 0.5×10⁷ EG7-GFP-hSLAMF7 cells into hind flank of mice. After 6-7 days, mice were randomized into the treatment groups when the average tumor volume reached 90-120 mm³. Antibody solutions were loaded into BD 28-gauge insulin syringes (VWR, Westchester, Pa.). 200-400 μl of antibodies formulated in PBS were administered intraperitoneally (i.p.) every three or four days, three to five doses and ranged from 1 to 10 mg/kg. Tumor growth was determined by measuring the tumor biweekly using Fowler Electronic Digital Caliper. The volume of the tumor was calculated with the following formula: L×W×H/2, where L (length) is the longest side of the tumor in the plane of the animal's back, W (width) is the longest measurement perpendicular to the length and in the plane of the animal's back, and H (height) is taken at the highest point perpendicular to the back of the animal. For each group, the number of tumor free (TF) mice was evaluated: tumor free mouse was defined as a mouse with a tumor of volume=0 for three consecutive measurements. Tumor growth delay (TGD) is the delay of a treated group to reach a selected volume compared to the control: TGD=T−C. T=median time (days) required for the treatment group tumors to reach a predetermined size. C=median time (days) required for the control group tumors to reach the same size.

### Elo-mIgG2a Serum Analysis

For characterization of pharmacokinetics of Elo-mIgG2a antibody, Balb/c mice were injected intraperitoneally with Elo-mIgG2a (1, 5 or 10 mg/kg) or mIgG2a (10 mg/kg). Blood samples were taken at 8 hours after the first dose, immediately before the second dose, immediately before the last dose, and 8 hours after the last dose and the sera were analyzed by ELISA. Nunc-Immuno MaxiSorp Microtiter plates were coated with HuLuc63 anti-idiotype monoclonal antibody in PBS overnight at 4 ° C. Sera samples were diluted 64,000-fold and Elo-mIgG2a was used as a standard. Plates were washed, incubated with mouse IgG2a-HRP at 1/1000 for 50 minutes at room temperature, and measured using TMB substrate. Concentrations of Elo-mIgG2a antibody in mouse serum samples were calculated from luminescence intensity as measured by M5 plate reader (Molecular Devices) using SOFTMAX® Pro.

### Isolation and Staining of Tumor Cells

Single cell suspension of tumor was prepared by dissociating tumor with the back of a syringe in a 24-well plate. Cell suspension was passed through 70-μm filter, pelleted, resuspended, and counted. Cells were then plated in 96-well plates with 1×10e6 cells per well for staining. Cells were treated with 2.4G2, which blocks Fc binding, and subsequently stained with anti-hSLAMF7 (clone 162.1, BioLegend) or anti-mIgG2b. Samples were analyzed on a FACSCanto flow cytometer (BD).

### Brief Description of the Sequence Listing

Incorporated herein by reference in its entirety is a Sequence Listing entitled, “12430-PCT_ST25.txt”, comprising SEQ ID NO:1 through SEQ ID NO:8, which includes the nucleic acid and/or amino acid sequences disclosed herein. The Sequence Listing has been submitted herewith in ASCII text format via EFS. The Sequence Listing was first created on Nov. 21, 2015, and is 10 KB in size.

### EXAMPLES

### Example 1

### Method for Cloning SLAMF7 cDNA into pFB Retroviral Vector

cDNA sequence from human SLAM family member 7 (hSLAMF7; synonyms: CS1-L) was cloned into retroviral vector encoding green fluorescent protein (GFP) (pFB-IRES-GFP, Stratagene).

The vector contains the murine leukemia retrovirus (MLV) packaging sequence and a multiple cloning site (MCS), flanked by the MLV long terminal repeat (LTR) regions.

The 5′ LTR functions as a strong promoter upon chromosomal integration of DNA. The pFB plasmid contains a cassette comprising an ECMV internal ribosome entry site (IRES) followed by a gene encoding GFP.

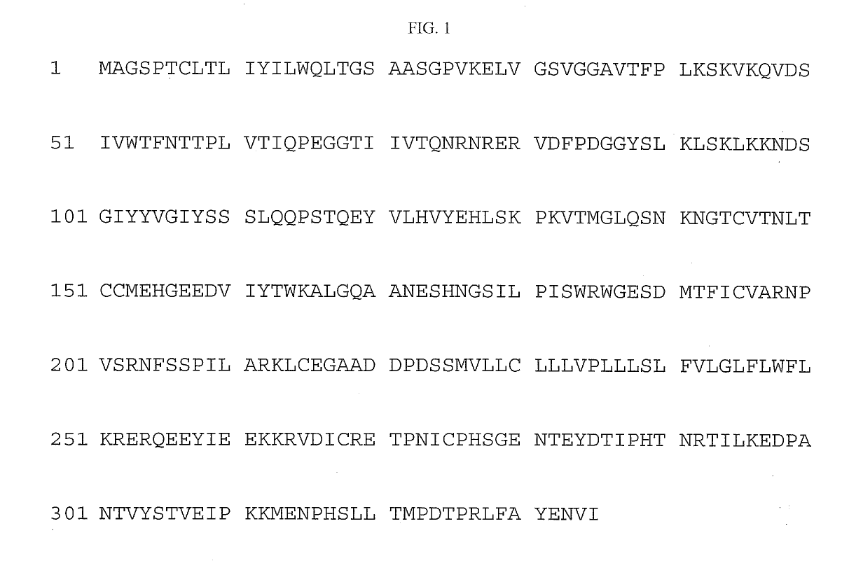

The cloned sequence of the encoded SLAMF7 protein sequence is provided in FIG. 1 (SEQ ID NO:7).

### Example 2

### Method for Generation of A20 Mouse Tumor Cell Line Expressing Human SLAMF7

The A20 mouse B lymphoma cell line was transduced with either retrovirus encoding GFP alone or with retrovirus encoding both GFP and hSLAMF7. A20-GFP and A20-hSLAMF7-GFP lines were sub-cloned, individual clones were picked and expanded in vitro. A20-GFP (clone D3) and A20-hSLAMF7-GFP (clone F11) were maintained in culture and expression of hSLAMF7 and GFP were assessed on day 58 to confirm the stability of hSLAMF7 expression.

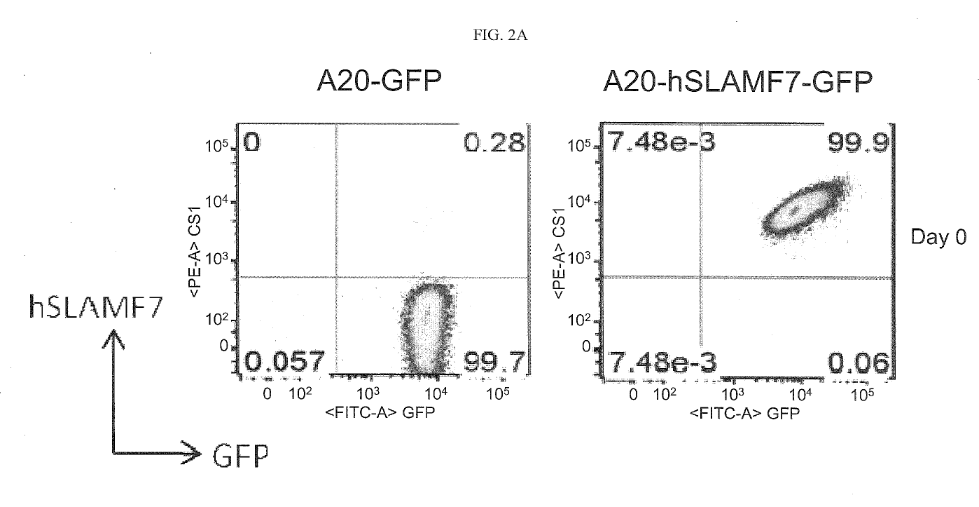

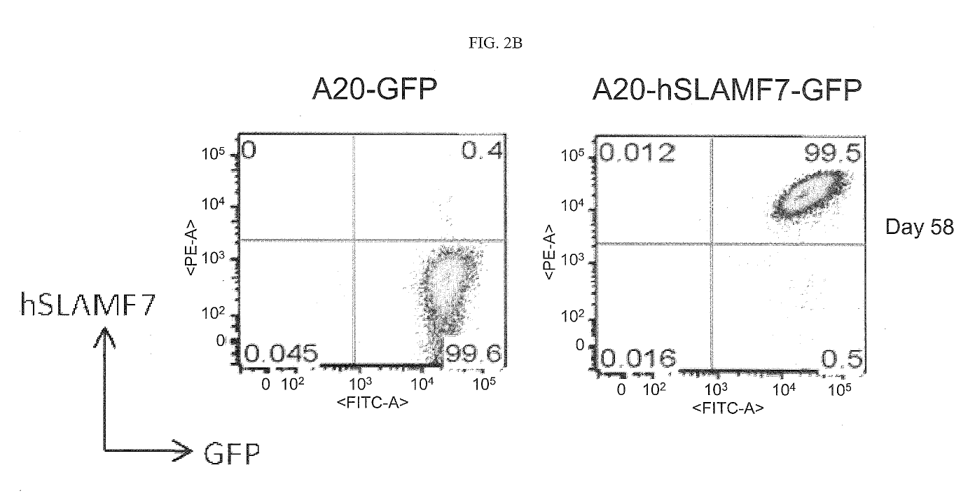

Cells were stained with PE-conjugated anti-human SLAMF7 (clone 162.1, BioLegend) and the frequency of cells staining positive for GFP and hSLAMF7 was determined. As shown in FIGS. 2A-B, A20 cell lines that stably express GFP and hSLAMF7 were obtained.

### Example 3

### Method for Determining Whether Elotuzumab Binds to Human SLAMF7 Expressed in A20 Cells

To determine whether hSLAMF7 expressed in A20 is recognized by Elotuzumab, A20-GFP and A20-hSLAMF7-GFP cells were stained with Elotuzumab. A20-GFP or A20-hSLAMF7-GFP cells were incubated with 6.25 ug/ml Elotuzumab (BMS), washed twice and incubated with anti-human IgG-PE secondary antibody. The frequency of cells staining positive for GFP and hSLAMF7 was determined using flow cytometry.

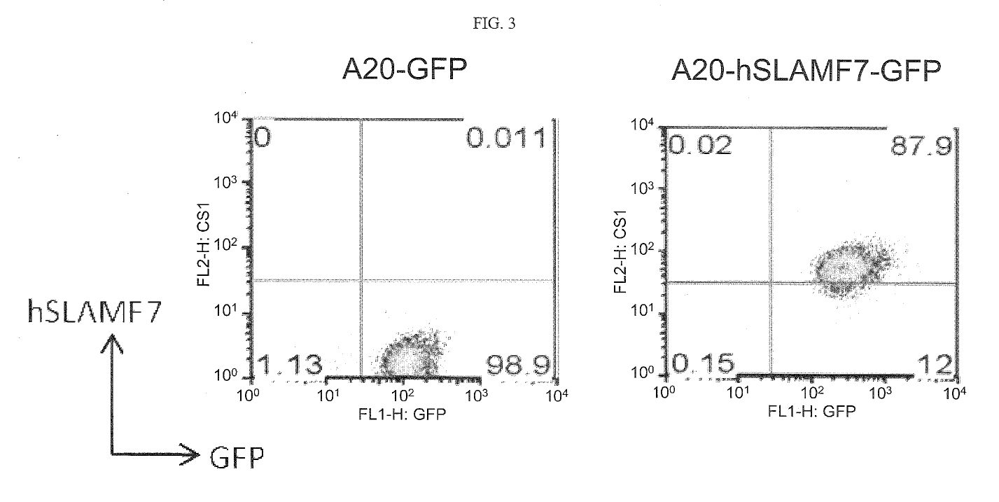

Surface staining, indicating Elotuzumab binding, was detected only in A20-hSLAMF7-GFP cells and not in A20-GFP cells as shown in FIG. 3.

### Example 4

### Method For Establishment of A20-hSLAMF7-GFP Tumor Model

This experiment was designed to determine whether A20-hSLAMF7-GFP cells engraft subcutaneously and grow in vivo.

Ten million A20-GFP or A20-hSLAMF7-GFP cells were engrafted in immunocompetent Balb/c mice. A20-hSLAMF7-GFP tumor growth was seen in 70% recipient mice ( 7/10) while A20-GFP tumor growth was seen in 100% recipient mice ( 10/10) (A).

Complete regression of the tumor was observed in ten to thirty percent of recipient mice, potentially due to immunogenicity of human SLAMF7 in Balb/c mice.

In order for A20-hSLAMF7-GFP tumor cells to be responsive to Elotuzumab treatment, it was important to determine that expression level of hSLAMF7 was maintained on A20-hSLAMF7-GFP cells when engrafted in mice.

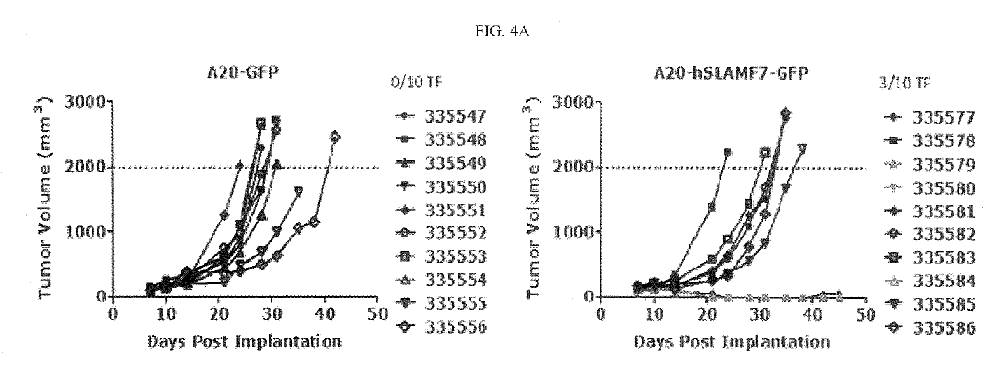

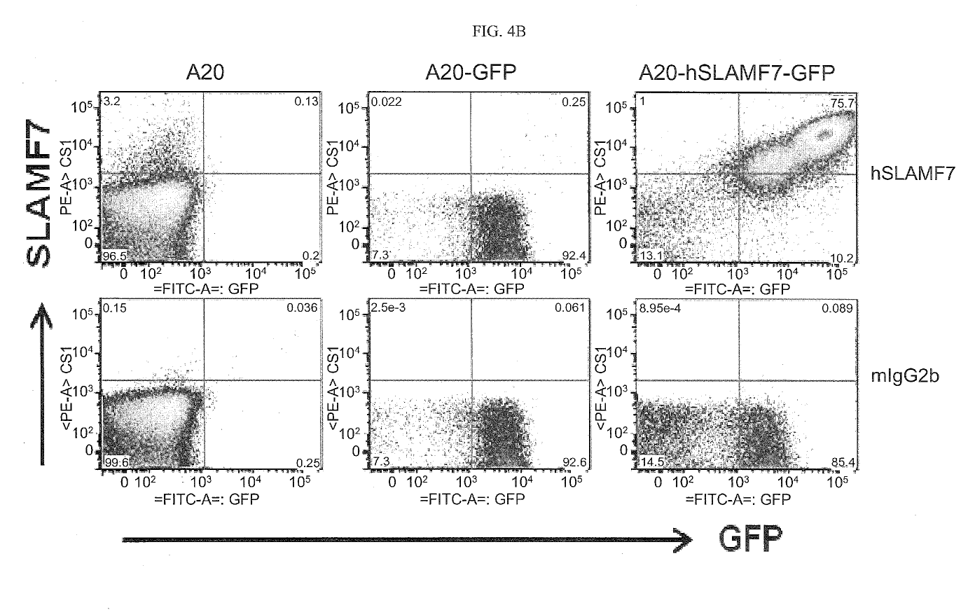

Tumors were established via subcutaneous injection of either 10⁷ A20-GFP or 10⁷ A20-hSLAMF7-GFP cells into the hind flank of Balb/c mice. Tumor growth was measured by digital caliper twice weekly (see FIG. 4A). Mice were euthanized when the tumors reached 2,000 mm³. Number of animals free of tumor by end of the experiment were designed tumor free (TF).

Cells isolated from A20-GFP or A20-hSLAMF7-GFP tumors were stained with anti-hSLAMF7 (clone 162.1, BioLegend) or mIgG2b isotype control antibody (MPC-11, BioLegend). Parental A20 cells maintained in culture were stained as a control. Samples were analyzed on a FACSCanto flow cytometer (BD) and percentage of cells positive for GFP and hSLAMF7 is shown.

A20-hSLAMF7-GFP and A20-GFP tumors were harvested from mice on day 45 after tumor cell inoculation and cells were stained for hSLAMF7 (see FIG. 4B). As shown, human SLAMF7 was expressed in A20-hSLAMF7-GFP cells isolated from mice but not in A20-GFP or parental A20 cells. Thus, A20-hSLAMF7-GFP cells grow in Balb/c mice and retain the surface expression of hSLAMF7.

### Example 5

### Method for Determining the Dose Response to Elo-G2A in the A20-hSLAMF7-GFP Tumor Model

To determine the potency of Elotuzumab in immunocompetent mice, the immunoglobulin heavy chain constant region of Elotuzumab was changed from human IgG1 to mouse IgG2a (mIgG2a). The Elotuzumab variant with the mIgG2a isotype is referred to Elo-mIgG2a.

The anti-tumoral activity of Elotuzumab against SLAMF7-expressing OPM2 tumors has been characterized in SCID mice at the dosage of 0.1, 0.5, 1 and 10 mg/kg (Tai, Y. et al., Blood, 112:1329-1337 (2008)). To determine the optimal dose of Elo-mIgG2a to be combined with anti-PD1, three doses were selected i.e., 1, 5 and 10 mg/kg.

Mice bearing A20-hSLAMF7-GFP tumors were randomized to different treatment groups when their tumors reached an average size of 180.1±87.3 mm³. Mice bearing A20-GFP tumors had tumors with the average size of 193.3±133.2 mm³; the treatment groups consisted of treatment with Elo-mIgG2a at doses 1, 5, and 10 mg/kg. The control group received mIgG2a control antibody (Bioxcell) at 10 mg/kg. Dosing was on days 14, 17, 21, 24, and 28. Experiment was terminated on day 59.

Anti-tumor activity of Elo-mIgG2a was tested in mice bearing A20-hSLAMF7-GFP tumors (G3, G4, and G5) or A20-GFP tumors (G1) which should not be responsive to Elo-mIgG2a activity since they do not express hSLAMF7. As a control for Elo-mIgG2a antibody, A20-hSLAMF7-GFP bearing mice were treated with anti-mouse IgG2a antibody (G2).

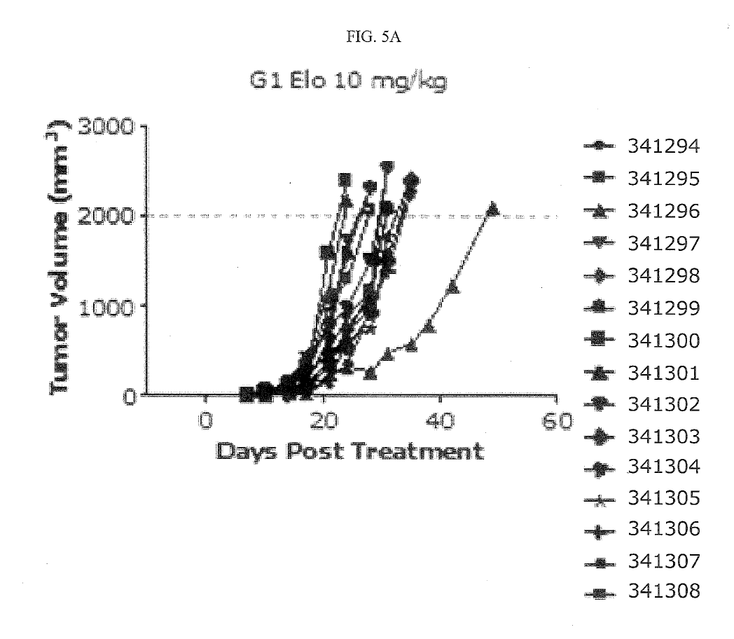

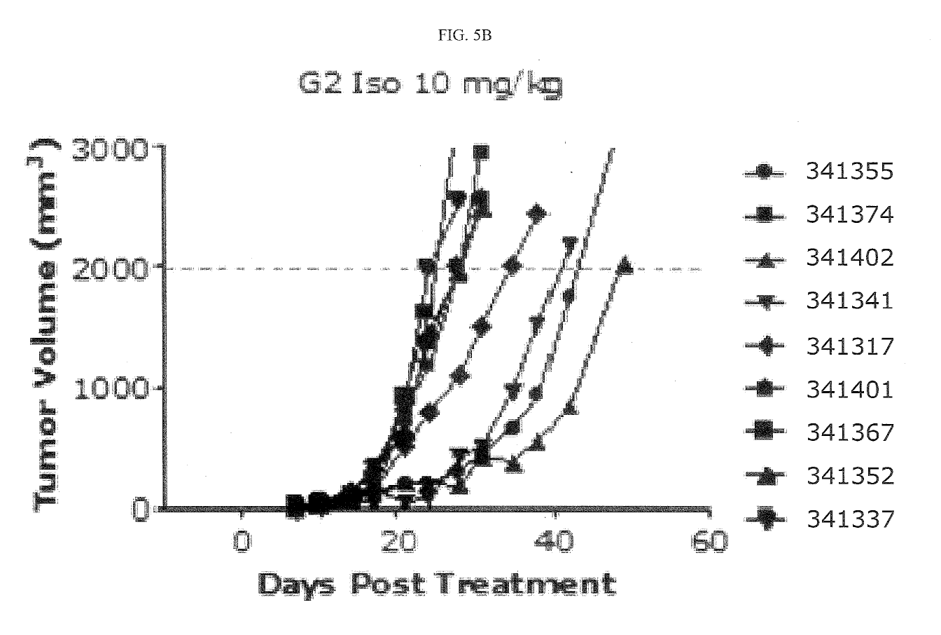

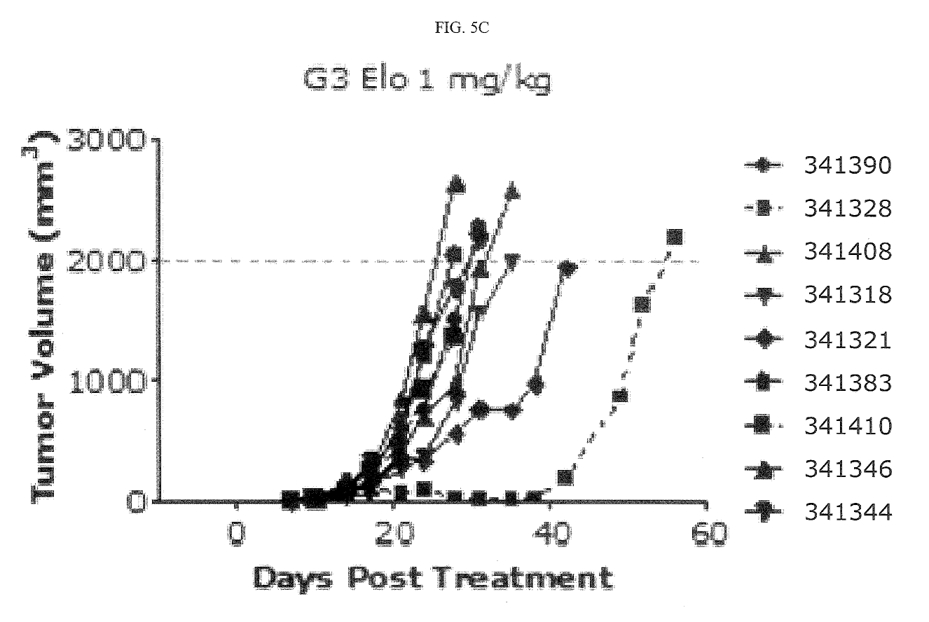

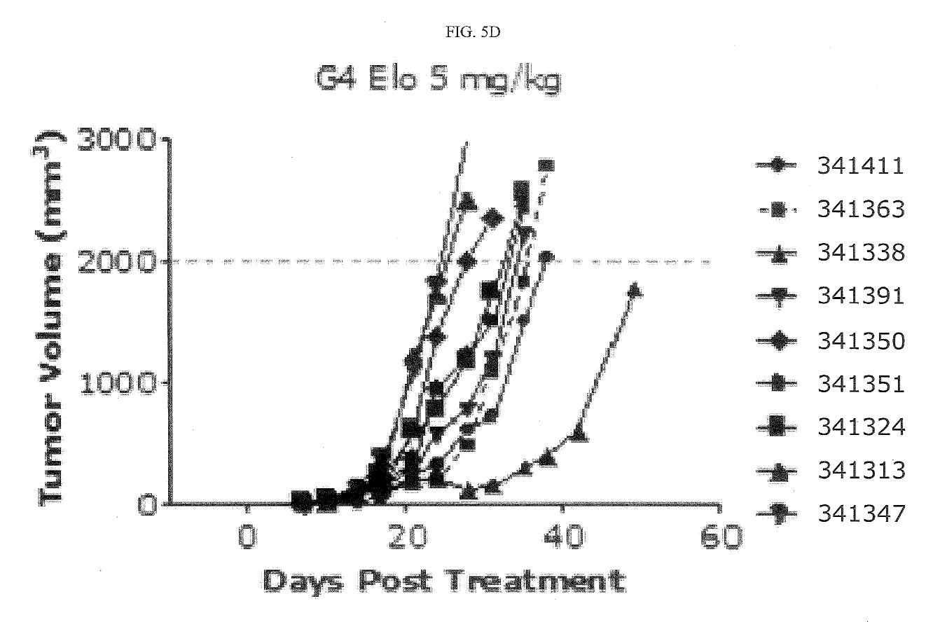

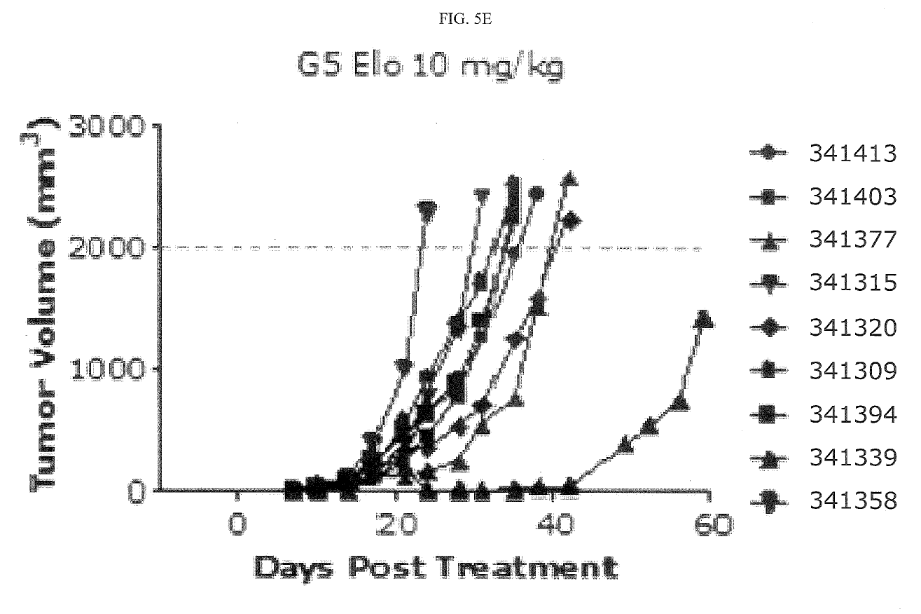

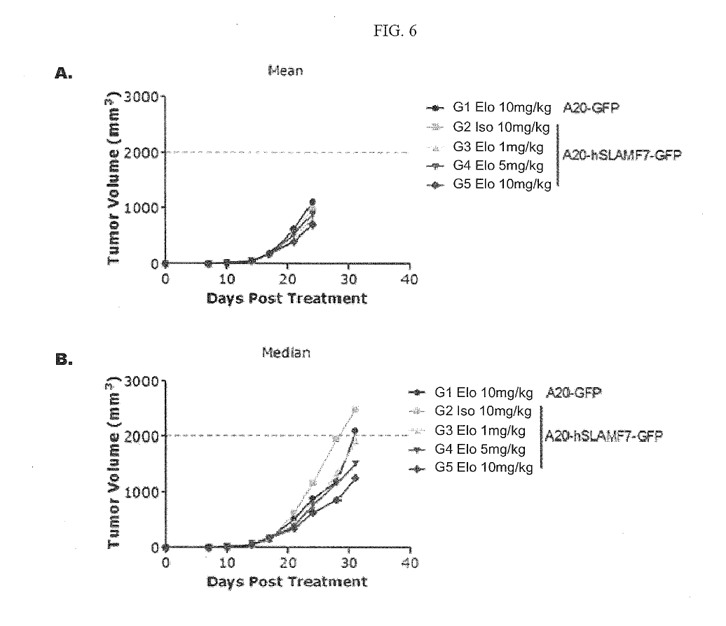

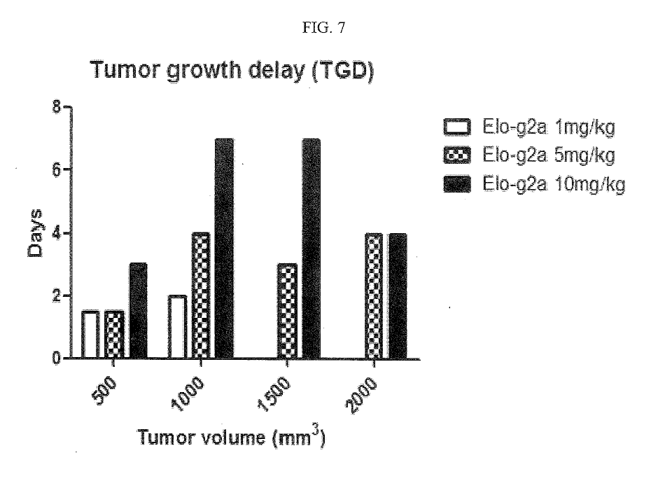

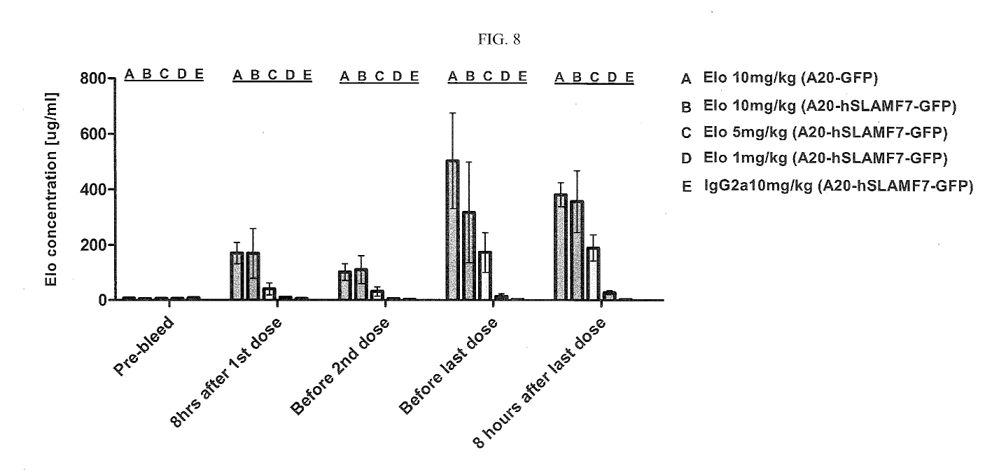

Tumor volumes of individual mice as shown in FIGS. 5A-E. The mean and median tumor volumes across five treatment groups are shown in FIG. 6. The tumor growth delay (TGD) of the different treatment groups relative to the control antibody (Iso 10 mg/kg) was calculated at 4 predetermined tumor volumes and is shown in FIG. 7. The TGD was calculated from 1 mg/kg (n=6), 5 mg/kg (n=8) and 10 mg/kg (n=8) mice.

Comparison of Elo-mIgG2a treated groups (G3, G4 and G5) with the control (G2) demonstrated that the dose of 10 mg/kg had stronger anti-tumoral activity compared to the doses of 1 or 5 mg/kg (see FIGS. 5A-E and 6). Moreover, tumor growth delay was increased in 10 mg/kg Elo-mIgG2a group compared to 1 mg/kg Elo-mIgG2a or isotype treated groups at all tumor volumes analyzed (see FIG. 7). Importantly, 10 mg/kg Elo-mIgG2a did not show anti-tumor activity in mice bearing A20-GFP tumors (G1) (see FIGS. 5A-E). In view of these results, 10 mg/kg of Elo-mIgG2a was selected to combine with anti-PD1 in the follow-up experiments.

### Example 6

### Method to Perform a Pharmacokinetic Analysis of Elo-mIgG2A in Tumor Bearing Balb/c Mice

Pharmacokinetic analysis of Elo-mIgG2a antibody was evaluated in tumor-bearing Balb/c mice.

Blood samples were collected at various time points from tumor bearing mice described in Example 5. Blood was collected prior to treatment (pre-bleed, day 14), at 8 hours after the first dose (day 15), immediately before the second dose (day 17), immediately before the last dose (day 28), and 8 hours after the last dose (day 29). N=3-9 mice/group.

Sera were analyzed by Enzyme-linked Immunosorbent Assay (ELISA). Serum samples were diluted 64,000-fold. Anti-idiotype monoclonal antibody to Elotuzumab (BMS) was used to capture Elo-mIgG2a in mouse serum samples. The captured Elo-mIgG2a was detected using anti-mouse IgG2a-HRP and measured using TMB substrate.

Measurement of Elo-mIgG2a concentrations in the serum samples obtained from mice with A20-hSLAMF7-GFP tumors showed that maximal anti-tumor activity correlated with 110±49 (before the second dose)-357±111 μg/mL (after the last dose) for the 10 mg/kg dose of Elo-mIgG2a while lower biological activity correlated with levels of 5±2-27±7 μg/mL for the 1 mg/kg dose of Elo-mIgG2a (see FIG. 8).

Serum levels of Elo-mIgG2a were similar in mice bearing A20-hSLAMF7-GFP and A20-GFP tumors (110±49-357±111 μg/mL vs. 102±30-381±43 μg/mL) for the 10 mg/kg dose of Elo-mIgG2a.

### Example 7

### Method to Determine Whether A20-hSLAMF7-GFP Tumor Cells Express PD-L1

To determine whether anti-PD1 antibody effects growth of A20-hSLAMF7-GFP tumors, the inventors first examined whether PD1 ligand, PD-L1, is expressed on A20 tumor cells.

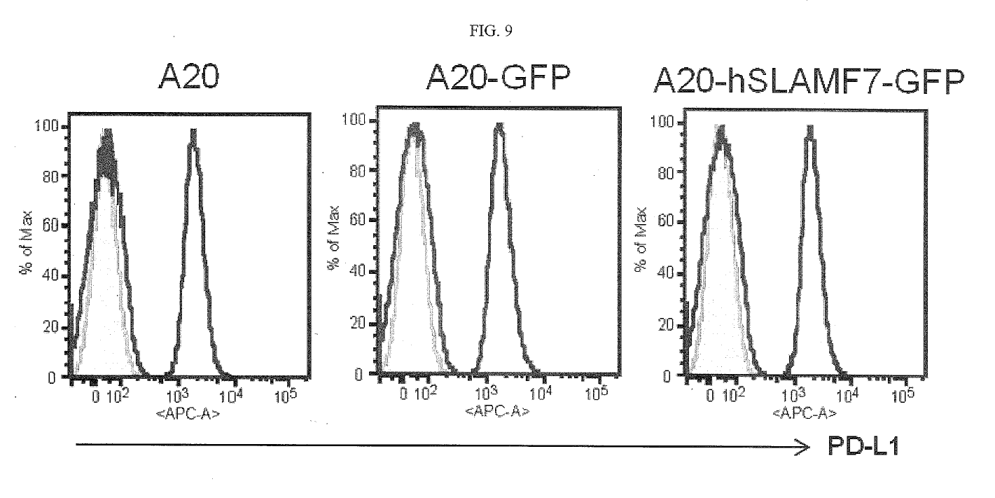

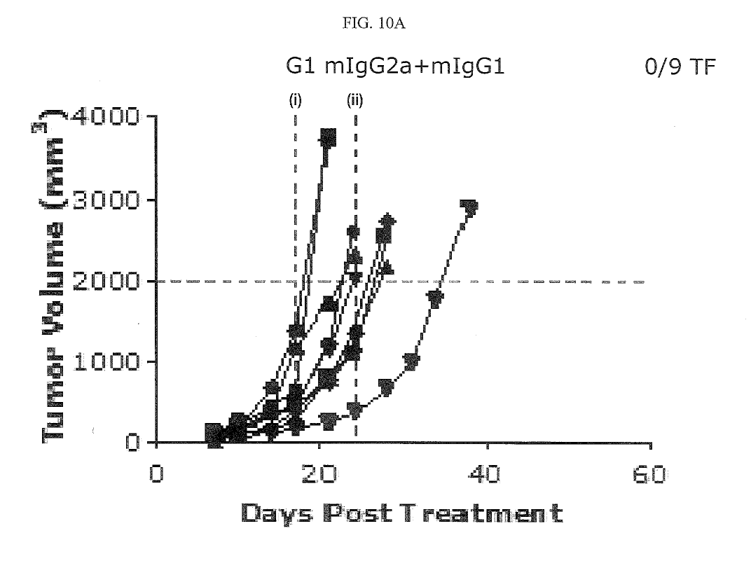

Flow cytometric analysis of PDL1 expression was determined and is shown in FIG. 9. Cells were unstained (light grey shaded line within first peak of histogram) or stained with either rat IgG2b (RTK4530, BioLegend) (dark outer line in first peak of histogram) or rat anti-mouse PD-L1 (10F.9G2, BioLegend) (dark line in second peak of histogram).

The results showed that both A20-hSLAMF7-GFP as well as A20-GFP cells express high level of PD-L1 which is similar to that in parental A20 cells (see FIG. 9).

These data provided a rationale for combination therapy with Elotuzumab that targets SLAMF7, a tumor antigen expressed by A20 cells and anti-PD1 antibody that activates T cells by blocking interaction between PD1 receptor on T cells and PD-L1 on A20 tumor cells.

### Example 8

### Methods for Assessing the Therapeutic Effect of Combining Elotuzumab with Anti-PD1 mAb in the A20-hSLAMF7-GFP Mouse Tumor Model

The therapeutic activity of Elo-mIgG2a in combination with blocking anti-PD1 antibody (PD-1-4H2-mg1-D265A) was tested in A20-hSLAMF7-GFP tumor model.

Elo-mIgG2a was used at 10 mg/kg and anti-PD1 antibody was tested at 3 mg/kg and 1 mg/kg to assess the therapeutic activity of combination regimens. Mice bearing A20-hSLAMF7-GFP tumors were randomized to different treatment groups at day 10 when their tumors reached an average size of 156.6±63.1 mm³. Elo-mIgG2a dosing was on days 10, 14, 17, 21 and 24 (5 doses). Anti-PD-1 or mIgG1 dosing was on days 10, 14 and 17 (3 doses). Experiment was terminated on day 44. Tumor volumes were measured biweekly. The number of tumor-free (TF) mice per group is shown for each group.

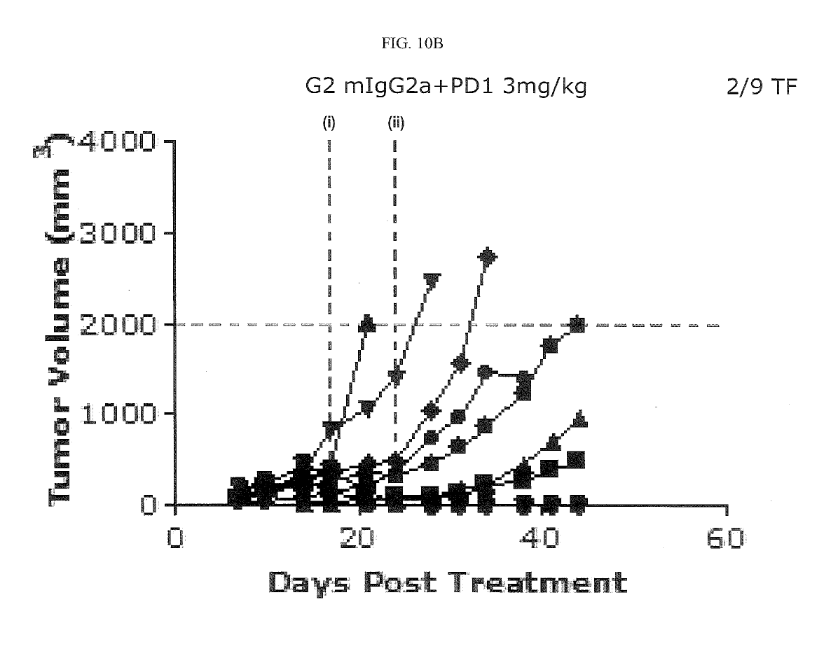

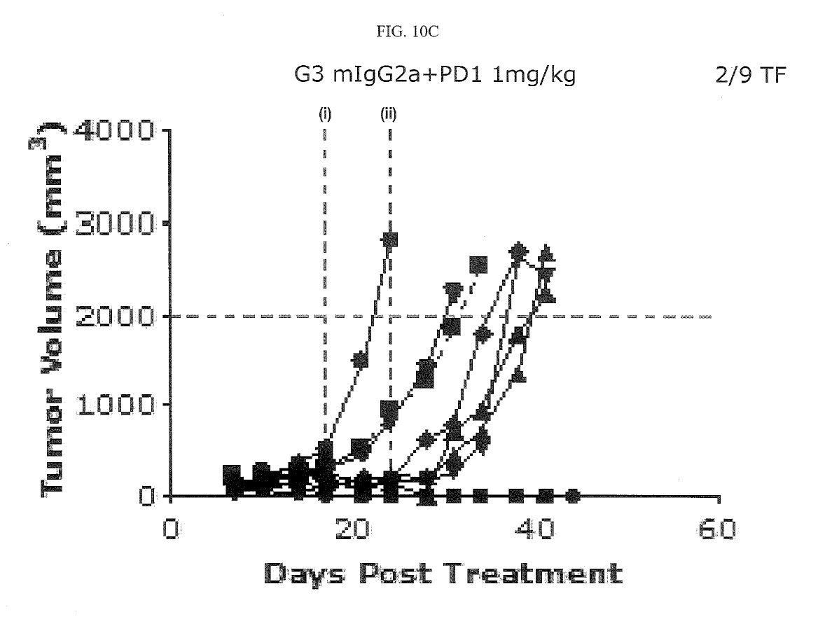

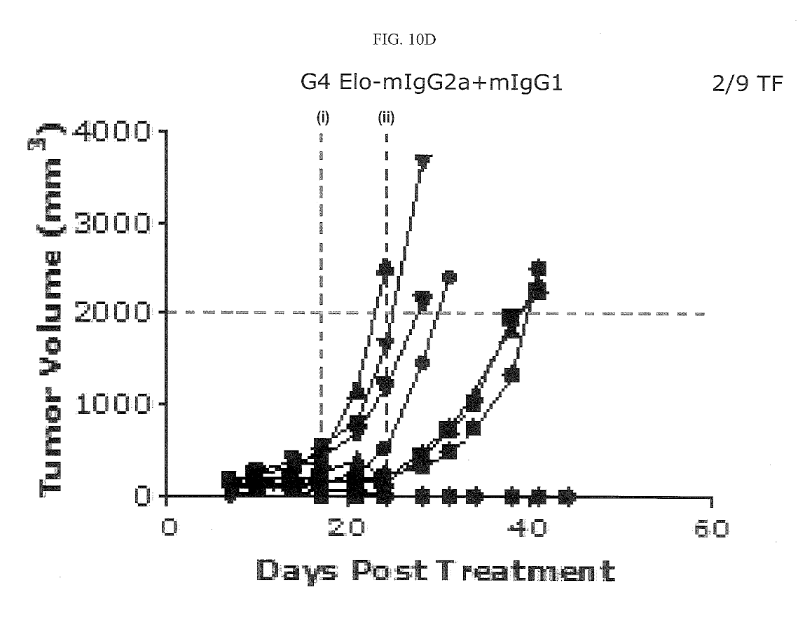

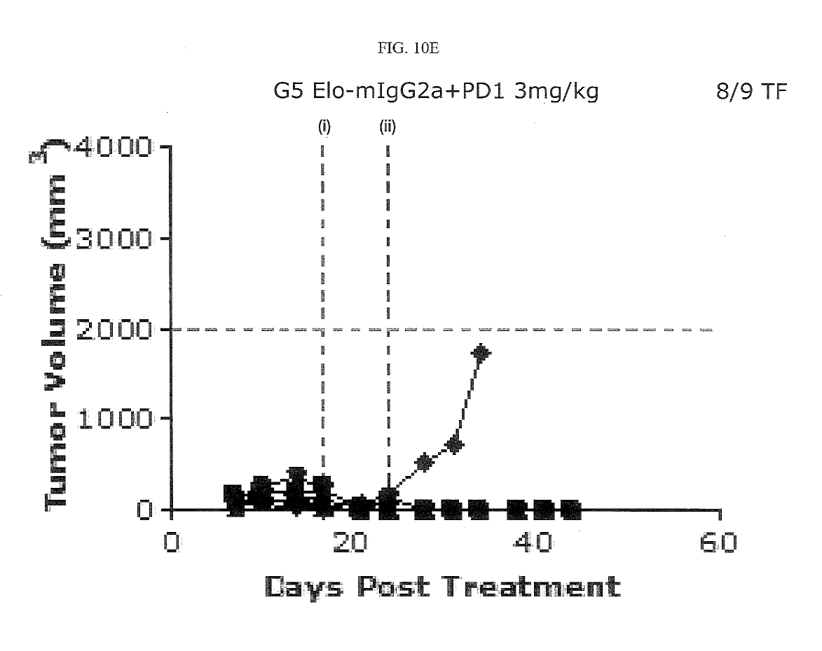

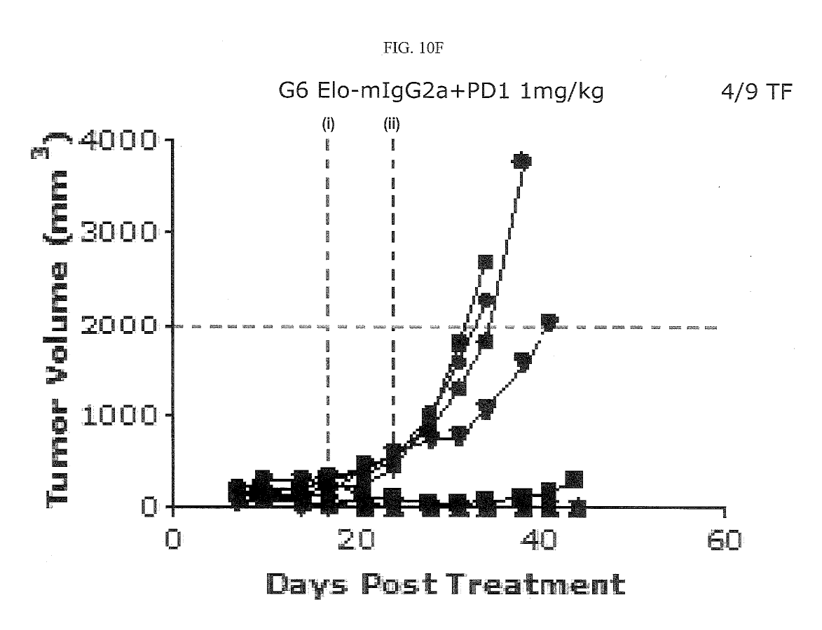

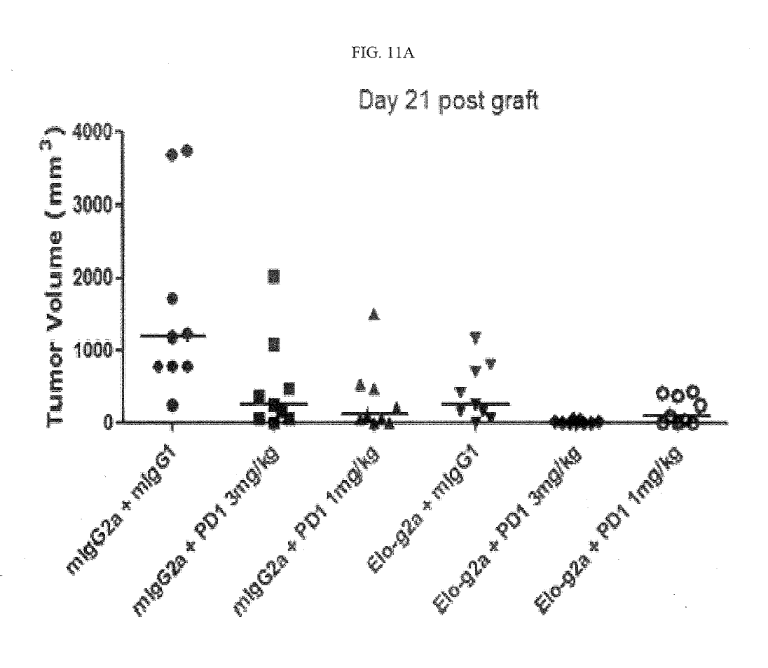

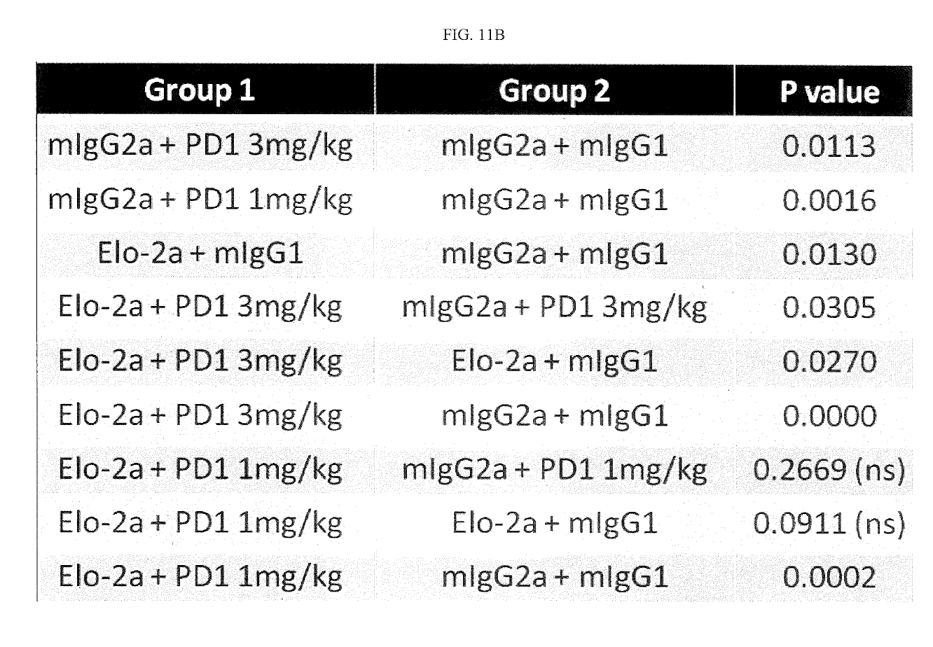

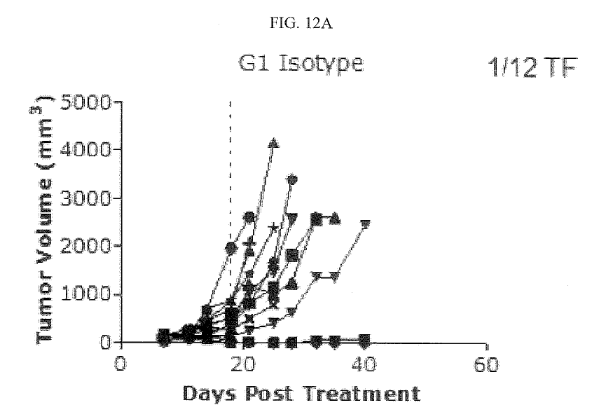

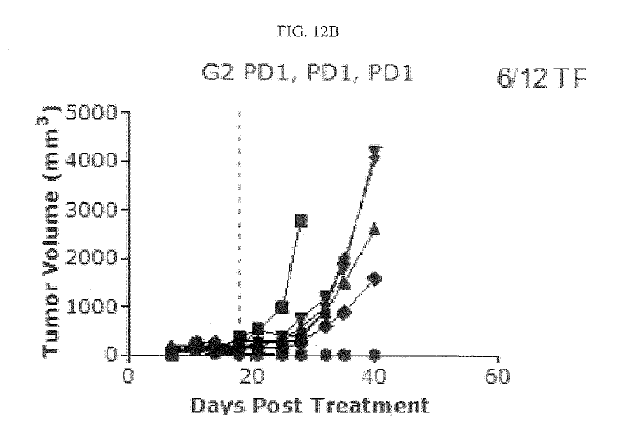

As shown in FIGS. 10A-F, the combined treatment of Elo-mIgG2a with anti-PD-1 resulted in a surprising increase in anti-tumor activity over Elo-mIgG2a or anti-PD-1 as a single agent. Specifically, analysis of curve profiles showed 2/9 tumor free mice in Elo-mIgG2a group (G4) compared to 0/9 tumor free mice in isotype treated control group (G1). Anti-PD1 treatment at either 3 mg/kg or 1 mg/kg resulted in 2/9 tumor free mice (G2, G3).

Addition of anti-PD1 antibody with Elo-mIgG2a resulted in strong, synergistic results. Specifically, the addition of the anti-PD1 antibody significantly improved the therapeutic activity of Elo-mIgG2a resulting in 8/9 tumor free mice when anti-PD1 was used at 3 mg/kg (G5) and 4/9 tumor free mice when anti-PD1 was used at 1 mg/kg (G6).

Comparison of the different treated groups at day 21 post tumor engraftment showed significantly decreased, median tumor volume for the combined treatment of Elo-mIgG2a with anti-PD-1, particularly when the anti-PD1 antibody was administered at a dose of 3 mg/kg.

As shown in FIG. 11B, statistical analysis performed at day 21 showed that Elo-mIgG2a+PD1 3 mg/kg combination resulted in a significant reduction in tumor volume compared to Elo-mIgG2a alone (p=0.0270) or to anti-PD1 3 mg/kg alone (p=0.0305).

### Conclusion

In view of the foregoing results, the combination of Elotuzumab with the IgG2a isotype (Elo-mIgG2a) was shown to have an anti-tumor activity against A20 tumor cells expressing hSLAMF7 in immunocompetent Balb/c mice. This activity was related to the level of Elo-mIgG2a observed in mouse sera. The combination of Elotuzumab and anti-PD1 antibody demonstrated synergistic anti-tumoral activity.

This study highlights the synergistic therapeutic efficacy of combination therapy with a cytotoxic antibody, Elotuzumab, that targets SLAMF7, a tumoral antigen expressed by multiple myeloma cells and an antibody that activates T cells by blocking interaction between PD1 receptor on T cells and PD-L1 on tumor cells. The combination of Elotuzumab with an anti-PD1 antibody demonstrated synergistic results when administered, particularly when Elotuzumab was administered at 10 mg/kg and the anti-PD1 mAb was administered at 3 mg/kg.

In non-clinical testing, the combination of Elotuzumab and nivolumab results in a safe and synergistic therapeutic effect, and does not result in a synergistic adverse event profile.

These results provide pre-clinical data to support the potential benefit of combining anti-SLAMF7 and anti-PD-1 antibodies in a clinical trial.

### Example 9

### Methods for Assessing the Therapeutic Effect of Combining Elotuzumab with Anti-PD1 mAb in the A20-hSLAMF7-GFP Mouse Tumor Model Using Either Concurrent or Sequential Administration

The effect of concurrent administration of anti-PD1 antibody and Elo-g2a in A20-hSLAMF7-GFP tumor model was investigated.

Different dosing regimens of anti-PD1 and Elo-g2a antibodies were studied in A20-hSLAMF7-GFP tumor model. Mice bearing A20-hSLAMF7-GFP tumors were randomized to different treatment groups at day 11 when their tumors had reached an average size of 179.6±59.5 mm³.

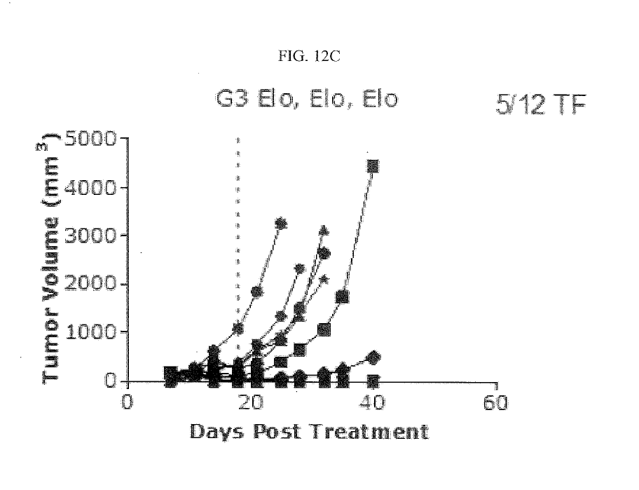

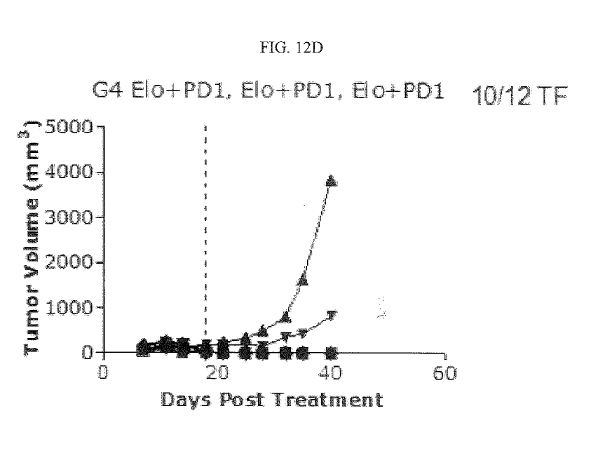

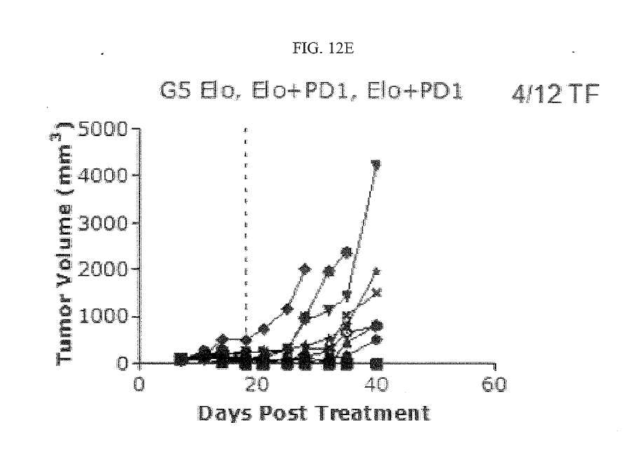

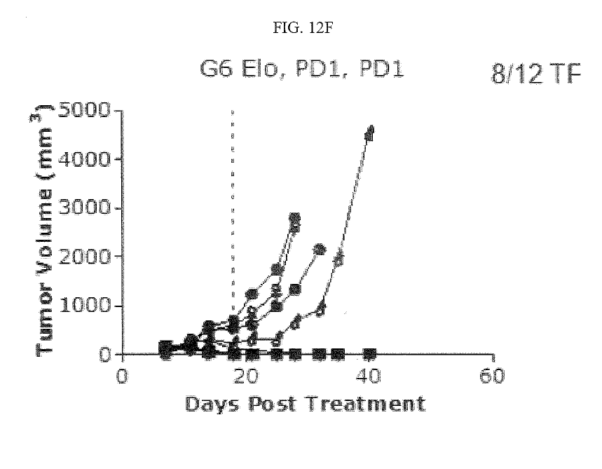

As shown in FIGS. 12A-F, when Elo-g2a and anti-PD1 were administered on the same day, complete regressions were observed in 10/12 mice (see FIG. 12D) compared to 6/12 in anti-PD1 (see FIG. 12B) and 5/12 in Elo-g2a (see FIG. 12C) treated groups, respectively. Synergistic effects of Elo-g2a and anti-PD1 resulted in fewer complete regressions in combination therapy groups with Elo-g2a and anti-PD1 administered sequentially (see FIG. 12D vs. FIGS. 12E and 12F). When independent dose of Elo-g2a was followed by either Elo-g2a and anti-PD1 combination (see FIG. 12E) or anti-PD1 alone (see FIG. 12F), 4/12 and 8/12 tumor free mice were observed, respectively.

### Conclusion

In view of the foregoing results, the combination therapy resulted in significant improvement in the anti-tumor effects when antibodies were dosed on the same day compared to sequential treatment suggesting that concurrent dosing may be preferred when this combination is administered in human clinical trials. The higher response levels observed between these experiments and the experiments outlined in Example 8 are likely attributable to the use of new lots of both the Elo and PD1 antibodies which had higher relative concentrations and thus, resulted in higher monotherapy response levels. Additional experiments designed to titrate the new Elo-g2a and anti-PD1 antibody lots to ensure they are functionally equivalent to the lots used in the Example 8 experiments are in progress.

These results provide pre-clinical data to support the potential benefit of combining anti-SLAMF7 and anti-PD-1 antibodies concurrently in a human clinical trial.

### Example 10

### Statistical Analysis Assessing the Therapeutic Effect of Combining Elotuzumab with Anti-PD1 mAb in the A20-hSLAMF7-GFP Tumor Model Mouse Model

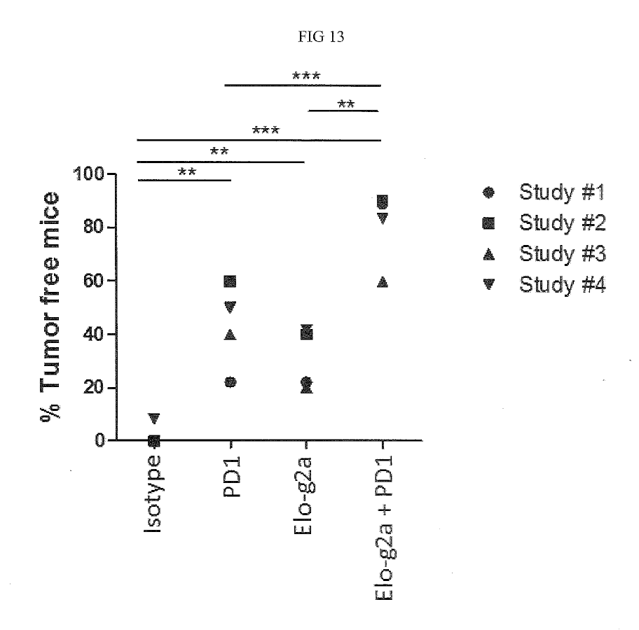

The therapeutic activity of Elo-g2a in combination with anti-PD1 antibody was evaluated across four independent studies. Binary logistic regression model was constructed to understand the differences between treatment groups in the proportion of mice that were tumor free at the end of the experiment. Overall effect of group: Wald chi-square (3) =29.64, p<0.0001. With isotype as reference, both Elo-g2a and anti-PD1 treated groups had far greater odds of being cancer free (Wald chi-square (1)=7.30, p=0.007, OR=18.48, 95% CI=2.23-153.37; Wald chi-square (1)=10.06, p=0.002, OR=30.26, 95%CI=3.68-248.85, respectively). The combination of Elo-g2a and anti-PD1 resulted in the greatest increase in the odds of being cancer free, Wald chi-square (1)=22.51, p<0.0001, OR=206.84, 95% CI=22.86-1871.88. To test whether the combination of Elo-g2a and anti-PD1 outperformed the single agents, the model was repeated, with combination as a reference group. Indeed, compared to the combination, the odds of either Elo-g2a or anti-PD1 groups being cancer free were far lower (Wald chi-square (1)=16.72, p<0.001, OR=0.09, 95% CI=0.03-0.28; Wald chi-square (1)=11.12, p=0.001, OR=0.15, 95% CI=0.05-0.45, respectively). The results of this statistical analysis are shown in FIG. 13 and show that at day 21, the Elo-mIgG2a+PD1 3 mg/kg combination resulted in a significant reduction in tumor volume compared to Elo-mIgG2a alone or to anti-PD1 3 mg/kg alone with p values ranging between <0.01 to <0.0001.

### Example 11

### Methods for Assessing the Therapeutic Effect of Combining Elotuzumab with Anti-PD1 mAb in an EG7 Lymphoma Tumor Model

The therapeutic activity of Elo-g2a in combination with anti-PD1 antibody was tested in the second syngeneic tumor model: the EG7 mouse lymphoma model.

Briefly, a stable EG7-hSLAMF7-GFP cell line was established using the same protocol described in Example 1 and elsewhere herein. Similar to the A20 transfected cell lines, the EG7-hSLAMF7-GFP cell line maintained high expression of SLAMF7 over time and also expressed high levels of PD-L1. Because subcutaneous administration of EG7 cells results in an aggressive solid lymphoma (Fransen, M. F. et al., Clin. Cancer Res., 19:5381-5389 (2013)), Elo-g2a and anti-PD1 antibodies were used at 10 mg/kg—a higher level of anti-PD1 antibody relative to the dose used in the A20 cell lines.

Mice bearing EG7-hSLAMF7-GFP tumors were randomized to different treatment groups at day 7 when their tumors reached an average size of 120.0±50.5 mm³. Elo-g2a and anti-PD1 dosing was on days 7, 10, and 14 (3 doses).

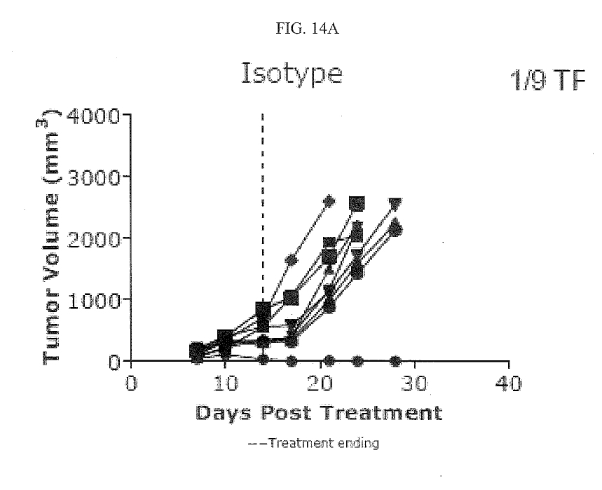

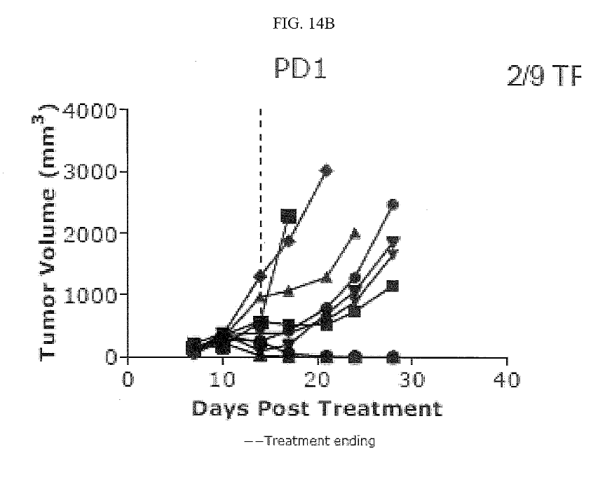

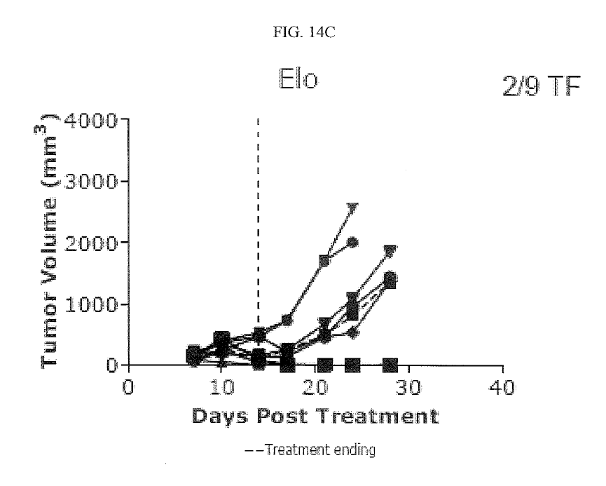

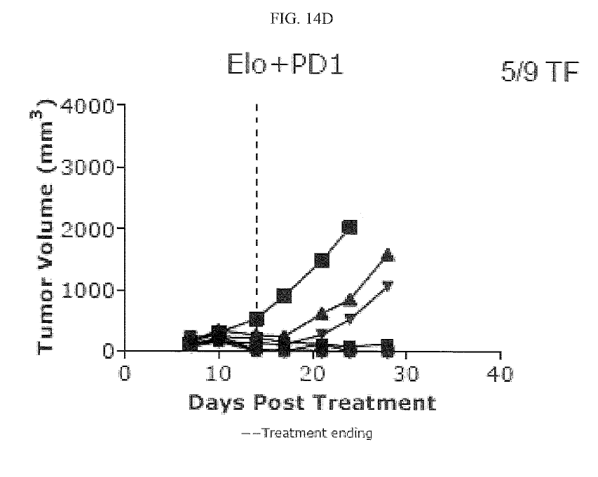

As shown in FIGS. 14A-D, analysis of curve profiles showed 2/9 tumor free mice in Elo-g2a group (G3) compared to 1/9 tumor free mice in isotype treated control group (G1). Anti-PD1 treatment resulted in 2/9 tumor free mice (G2). Addition of anti-PD1 antibody significantly improved the therapeutic activity of Elo-g2a resulting in overall tumor growth inhibition and 5 out of 9 mice being tumor free (G4).

### Conclusion

Overall, Elotuzumab comprising an IgG2a isotype (Elo-g2a) was shown to have an anti-tumor activity against both A20 and EG7 tumor cells expressing hSLAMF7 in immunocompetent Balb/c (A20 model) or C57BL/6 (EG7 model) mice. This activity was directly related to the level of Elo-g2a observed in mouse sera. The combination of Elotuzumab and anti-PD1 antibody demonstrated a synergistic anti-tumoral activity. Combination therapy resulted in significant improvement in the anti-tumor effects when antibodies were dosed on the same day compared to sequential treatment suggesting that concurrent dosing could be selected in human clinical trials. Overall, these studies highlight the synergistic therapeutic efficacy of combination therapy with a cytotoxic antibody that targets SLAMF7, Elotuzumab, and an antibody that activates T cells by blocking interaction between PD1 receptor on T cells and PD-L1 on tumor cells.

These results further provide pre-clinical data to support the potential benefit of combining anti-SLAMF7 and anti-PD-1 antibodies concurrently in a human clinical trial.

The entire disclosure of each document cited (including patents, patent applications, journal articles, abstracts, laboratory manuals, books, GENBANK® Accession numbers, SWISS-PROT® Accession numbers, or other disclosures) in the Background of the Invention, Detailed Description, Brief Description of the Figures, and Examples is hereby incorporated herein by reference in their entirety. Further, the hard copy of the Sequence Listing submitted herewith, in addition to its corresponding Computer Readable Form, are incorporated herein by reference in their entireties.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

What is claimed is:

1. A method for treating a patient with cancer comprising the administration of a combination therapeutic regiment comprising: (i) a therapeutically effective amount of an anti-PD1 antibody; and (ii) a therapeutically effective amount of an anti-CS1 antibody, wherein said combination results in the synergistic reduction in tumor burden, tumor regression, and/or tumor development of said cancer.

2. The method of claim 1, wherein said cancer is selected from the group consisting of: myeloma, multiple myeloma, and smoldering myeloma.

3. The method according to claim 1, wherein said anti-PD1 antibody is nivolumab.

4. The method of claim 1, 2, or 3, wherein said anti-CS1 antibody is elotuzumab.

5. The method of claim 1, wherein said anti-PD1 antibody is administered at a dosage of about 0.1-3 mg/kg, and said anti-CS1 antibody is administered at a dosage of about 0.1-1 mg/kg.

6. The method of claim 1, wherein said anti-PD1 antibody is administered at a dosage of about 1 mg/kg, and said anti-CS1 antibody is administered at a dosage of about 10 mg/kg.

7. The method of claim 1, wherein said anti-PD1 antibody is administered at a dosage of about 3 mg/kg, and said anti-CS1 antibody is administered at a dosage of about 10 mg/kg.

8. The method of claim 1, wherein said anti-PD1 antibody is administered at a dosage of about 0.1-3 mg/kg, and said anti-CS1 antibody is administered at a dosage of about 1 mg/kg or 10 mg/kg.

9. The method of claim 1, wherein said cancer is selected from the group consisting of: lymphoma, non-Hodgkin's lymphomas (NHL), chronic lymphocytic leukemia, follicular lymphoma, mantle cell lymphoma and diffuse large B-cell lymphoma.
